SEQUENCE LISTING (35 sequences)

SEQ ID NO: 1 - protein, 32 aa, synthetic construct
  REGION 1..32: note = Seq. No 1a
KKQAQRKRHKLNRKERGHKSPSEQRRSELWHA

SEQ ID NO: 2 - protein, 32 aa, synthetic construct
  SITE 4: note = side chain-to-side chain crosslinking amino acid
  SITE 11: note = side chain-to-side chain crosslinking amino acid
  REGION 1..32: note = Seq. No 1b
KKQXQRKRHKXNRKERGHKSPSEQRRSELWHA

SEQ ID NO: 3 - protein, 32 aa, synthetic construct
  SITE 7: note = side chain-to-side chain crosslinking amino acid
  SITE 11: note = side chain-to-side chain crosslinking amino acid
  REGION 1..32: note = Seq. No 1c
KKQAQRXRHKXNRKERGHKSPSEQRRSELWHA

SEQ ID NO: 4 - protein, 32 aa, synthetic construct
  SITE 11: note = side chain-to-side chain crosslinking amino acid
  SITE 15: note = side chain-to-side chain crosslinking amino acid
  REGION 1..32: note = Seq. No 1d
KKQAQRKRHKXNRKXRGHKSPSEQRRSELWHA

SEQ ID NO: 5 - protein, 32 aa, synthetic construct
  SITE 24: note = side chain-to-side chain crosslinking amino acid
  SITE 28: note = side chain-to-side chain crosslinking amino acid
  REGION 1..32: note = Seq. No 1e
KKQAQRKRHKLNRKERGHKSPSEXRRSXLWHA

SEQ ID NO: 6 - protein, 32 aa, synthetic construct
  SITE 28: note = side chain-to-side chain crosslinking amino acid
  SITE 32: note = side chain-to-side chain crosslinking amino acid
  REGION 1..32: note = Seq. No 1f
KKQAQRKRHKLNRKERGHKSPSEQRRSXLWHX

SEQ ID NO: 7 - protein, 32 aa, synthetic construct
  SITE 4: note = side chain-to-side chain crosslinking amino acid
  SITE 11: note = side chain-to-side chain crosslinking amino acid
  SITE 24: note = side chain-to-side chain crosslinking amino acid
  SITE 28: note = side chain-to-side chain crosslinking amino acid
  REGION 1..32: note = Seq. No 1g
KKQXQRKRHKXNRKERGHKSPSEXRRSXLWHA

SEQ ID NO: 8 - protein, 32 aa, synthetic construct
  SITE 4: note = side chain-to-side chain crosslinking amino acid
  SITE 11: note = side chain-to-side chain crosslinking amino acid
  SITE 28: note = side chain-to-side chain crosslinking amino acid
  SITE 32: note = side chain-to-side chain crosslinking amino acid
  REGION 1..32: note = Seq. No 1h
KKQXQRKRHKXNRKERGHKSPSEQRRSXLWHX

SEQ ID NO: 9 - protein, 32 aa, synthetic construct
  SITE 7: note = side chain-to-side chain crosslinking amino acid
  SITE 11: note = side chain-to-side chain crosslinking amino acid
  SITE 24: note = side chain-to-side chain crosslinking amino acid
  SITE 28: note = side chain-to-side chain crosslinking amino acid
  REGION 1..32: note = Seq. No 1i
KKQAQRXRHKXNRKERGHKSPSEXRRSXLWHA

SEQ ID NO: 10 - protein, 32 aa, synthetic construct
  SITE 7: note = side chain-to-side chain crosslinking amino acid
  SITE 11: note = side chain-to-side chain crosslinking amino acid
  SITE 28: note = side chain-to-side chain crosslinking amino acid
  SITE 32: note = side chain-to-side chain crosslinking amino acid
  REGION 1..32: note = Seq. No 1k
KKQAQRXRHKXNRKERGHKSPSEQRRSXLWHX

SEQ ID NO: 11 - protein, 32 aa, synthetic construct
  SITE 11: note = side chain-to-side chain crosslinking amino acid
  SITE 15: note = side chain-to-side chain crosslinking amino acid
  SITE 24: note = side chain-to-side chain crosslinking amino acid
  SITE 28: note = side chain-to-side chain crosslinking amino acid
  REGION 1..32: note = Seq. No 1l
KKQAQRKRHKXNRKXRGHKSPSEXRRSXLWHA

SEQ ID NO: 12 - protein, 32 aa, synthetic construct
  SITE 11: note = side chain-to-side chain crosslinking amino acid
  SITE 15: note = side chain-to-side chain crosslinking amino acid
  SITE 28: note = side chain-to-side chain crosslinking amino acid
  SITE 32: note = side chain-to-side chain crosslinking amino acid
  REGION 1..32: note = Seq. No 1m
KKQAQRKRHKXNRKXRGHKSPSEQRRSXLWHX

SEQ ID NO: 13 - protein, 16 aa, synthetic construct
  REGION 1..16: note = Seq. No 1n
KKQAQRKRHKLNRKER

SEQ ID NO: 14 - protein, 16 aa, synthetic construct
  CROSSLNK 11..15: note = crosslinked amino acids X-X
  REGION 1..16: note = Synthetic peptide
KKQAQRKRHKXNRKXR

SEQ ID NO: 15 - RNA, 22 nt, Homo sapiens
  source 1..22: mol_type = other RNA
  modified_base 1: mod_base = OTHER; note = 5-terminal phosphate
tagcttatcagactgatgttga

SEQ ID NO: 16 - RNA, 20 nt, Homo sapiens
  source 1..20: mol_type = other RNA
  modified_base 1: mod_base = OTHER; note = 5-terminal phosphate
aacaccagtcgatgggctgt

SEQ ID NO: 17 - RNA, 21 nt, synthetic construct
  source 1..21: mol_type = other RNA
  modified_base 1: mod_base = OTHER; note = labeled with Cyanine 5: 2-((1E,3E)-5-((E)-1-(3-(lam bda-oxidaneyl)propyl)-3,3-dimethylindolin-2-ylidene)penta- 1,3-dien-1-yl)-1-(3hydroxypropyl)-3,3-dimethyl-3H-indol-1- ium
  modified_base 20: mod_base = OTHER; note = thymine
  modified_base 21: mod_base = OTHER; note = thymine
  misc_feature 1..21: note = Cy5 siRNA 5'
cttacgctgagtacttcgatt

SEQ ID NO: 18 - RNA, 21 nt, synthetic construct
  source 1..21: mol_type = other RNA
  modified_base 1: mod_base = OTHER; note = 5-terminal phosphate
  modified_base 20: mod_base = OTHER; note = thymine
  modified_base 21: mod_base = OTHER; note = thymine
  misc_feature 1..21: note = Cy5 siRNA 3'
tcgaagtactcagcgtaagtt

SEQ ID NO: 19 - protein, 33 aa, synthetic construct
  SITE 1: note = acetylation of N terminus
  REGION 1..33: note = wt33 peptide
KKQAQRKRHKLNRKERGHKSPSEQRRSELWHAR

SEQ ID NO: 20 - protein, 19 aa, synthetic construct
  SITE 1: note = acetylation of N terminus
  REGION 1..19: note = Peptide 1
MNQKKQAQRKRHKLNRKER

SEQ ID NO: 21 - protein, 19 aa, synthetic construct
  SITE 1: note = bAla
  SITE 1: note = bAla is attached through the linker 3-mercaptopropionic acid to another amino acid sequence
  REGION 1..19: note = Peptide
ANQKKQAQRKRHKLNRKER

SEQ ID NO: 22 - protein, 19 aa, synthetic construct
  SITE 1: note = acetylation of N terminus
  SITE 14: note = (S)-2-(4-pentenyl)alanine
  SITE 18: note = (S)-2-(4-pentenyl)alanine
  REGION 1..19: note = Peptide 2
MNQKKQAQRKRHKANRKAR

SEQ ID NO: 23 - protein, 19 aa, synthetic construct
  SITE 1: note = bAla
  SITE 1: note = bAla is attached through the linker 3-mercaptopropionic acid to another amino acid sequence
  SITE 14: note = (S)-2-(4-pentenyl)alanine
  SITE 18: note = (S)-2-(4-pentenyl)alanine
  REGION 1..19: note = Peptide
ANQKKQAQRKRHKANRKAR

SEQ ID NO: 24 - protein, 30 aa, synthetic construct
  SITE 1: note = acetylation of N terminus
  REGION 1..30: note = Peptide 3
PLHEIIRKLERMNQKKQAQRKRHKLNRKER

SEQ ID NO: 25 - protein, 16 aa, synthetic construct
  SITE 4: note = 2-(7-octenyl)alanine
  SITE 11: note = 2-(4-pentenyl)alanine
  REGION 1..16: note = Seq. No 1o
KKQAQRKRHKANRKER

SEQ ID NO: 26 - protein, 16 aa, synthetic construct
  SITE 7: note = 2-(4-pentenyl)alanine
  SITE 11: note = 2-(4-pentenyl)alanine
  REGION 1..16: note = Seq. No 1p
KKQAQRARHKANRKER

SEQ ID NO: 27 - protein, 16 aa, synthetic construct
  SITE 11: note = 2-(4-pentenyl)alanine
  SITE 15: note = 2-(4-pentenyl)alanine
  REGION 1..16: note = Seq. No 1q
KKQAQRKRHKANRKAR

SEQ ID NO: 28 - protein, 28 aa, synthetic construct
  SITE 24: note = 2-(4-pentenyl)alanine
  SITE 28: note = 2-(4-pentenyl)alanine
  REGION 1..28: note = Seq. No 1r
KKQAQRKRHKLNRKERGHKSPSEARRSA

SEQ ID NO: 29 - protein, 27 aa, synthetic construct
  REGION 1..27: note = Seq. No 1s
KKQAQRKRHKLNRKERGHKSPSEQRRS

SEQ ID NO: 30 - protein, 28 aa, synthetic construct
  SITE 4: note = 2-(7-octenyl)alanine
  SITE 11: note = 2-(4-pentenyl)alanine
  SITE 24: note = 2-(4-pentenyl)alanine
  SITE 28: note = 2-(4-pentenyl)alanine
  REGION 1..28: note = Seq. No 1t
KKQAQRKRHKANRKERGHKSPSEARRSA

SEQ ID NO: 31 - protein, 27 aa, synthetic construct
  SITE 4: note = 2-(7-octenyl)alanine
  SITE 11: note = 2-(4-pentenyl)alanine
  REGION 1..27: note = Seq. No 1u
KKQAQRKRHKANRKERGHKSPSEQRRS

SEQ ID NO: 32 - protein, 28 aa, synthetic construct
  SITE 7: note = 2-(4-pentenyl)alanine
  SITE 11: note = 2-(4-pentenyl)alanine
  SITE 24: note = 2-(4-pentenyl)alanine
  SITE 28: note = 2-(4-pentenyl)alanine
  REGION 1..28: note = Seq. No 1v
KKQAQRARHKANRKERGHKSPSEARRSA

SEQ ID NO: 33 - protein, 27 aa, synthetic construct
  SITE 7: note = 2-(4-pentenyl)alanine
  SITE 11: note = 2-(4-pentenyl)alanine
  REGION 1..27: note = Seq. No 1w
KKQAQRARHKANRKERGHKSPSEQRRS

SEQ ID NO: 34 - protein, 28 aa, synthetic construct
  SITE 11: note = 2-(4-pentenyl)alanine
  SITE 15: note = 2-(4-pentenyl)alanine
  SITE 24: note = 2-(4-pentenyl)alanine
  SITE 28: note = 2-(4-pentenyl)alanine
  REGION 1..28: note = Seq. No 1x
KKQAQRKRHKANRKARGHKSPSEARRSA

SEQ ID NO: 35 - protein, 27 aa, synthetic construct
  SITE 11: note = 2-(4-pentenyl)alanine
  SITE 15: note = 2-(4-pentenyl)alanine
  REGION 1..27: note = Seq. No 1y
KKQAQRKRHKANRKARGHKSPSEQRRS

(19) United States
(12) Patent Application Publication
(10) Pub. No.: US 2026/0209277 A1
(43) Pub. Date: Jul. 23, 2026

(54) COMPOUNDS FOR RNA STABILISATION AND DELIVERY

(71) Applicant: STICHTING VU, Amsterdam (NL)

(72) Inventors: Tom N. GROSSMANN, Amsterdam (NL); Niall M. MCLOUGHLIN, Amsterdam (NL); Saskia NEUBACHER, Amsterdam (NL)

(21) Appl. No.: 19/116,072

(22) PCT Filed: Sep. 29, 2023
(86) PCT No.: PCT/NL2023/050512

§ 371 Date: Mar. 27, 2025

(30) Foreign Application Priority Data
Sep. 29, 2022 (NL) 2033185

(51) Int. Cl. C07K 14/00; C07K 19/00; C12N 15/113

(52) U.S. Cl. CPC C07K 14/00; C07K 19/00; C12N 15/113; C12N 2310/141; C12N 2310/531

(57) ABSTRACT

The present invention relates to a peptide-based compound for complexing and stabilizing a double stranded oligonucleotide, the compound comprising a structure p-x-b-x′-p′; wherein: i. p and p′ each refer to an oligonucleotide-binding motif; ii. x and x′ each refer to an optional linker motif, and iii. b is a linking motif coupling the oligonucleotide-binding motif to form a dimerized form, wherein motif p and p′ each independently represent a peptide chain having the following fragment comprising a contiguous sequence of at least 14 amino acid residues, and having the following general sequence (I), wherein the N-terminal position 1 is located on the left side: wherein “v” represents a variable amino acid residue position, and wherein “+” represents a positively charged amino acid residue.

### CROSS-REFERENCE TO RELATED APPLICATIONS

This application is a national phase application of PCT Application No. PCT/NL2023/050512, filed on 29 Sep. 2023, which claims the benefit and priority to Netherlands Patent Application No. 2033185, filed on 29 Sep. 2022. The entire disclosures of the applications identified in this paragraph are incorporated herein by references.

The present invention relates to synthetic modified peptides useful for increasing the stability of ribonucleic acid (RNA) and the delivery efficiency of RNA cargos to target eukaryotic cells. More specifically, the present invention relates to synthetic peptides and peptide-based shuttle agents for cellular delivery of siRNA for therapeutic, biotechnological and diagnostic applications, and/or stabilization, and support of cellular delivery of RNA containing double stranded regions for therapeutic, biotechnological and diagnostic applications.

### BACKGROUND OF THE INVENTION

Ribonucleic acid (RNA) is an essential biopolymer that acts as the key intermediate in the transmission of genetic information into proteins. Recently, advances in next-generation sequencing and transcriptomics have revealed that RNA also plays many unprecedented, functional roles in the regulation of cellular processes with disease-associated implications. Hence, significant interest has grown in the design of RNA binding molecules that can be used to interrogate biological functions. However, the progression of RNA-based cellular applications in molecular therapy and diagnostics has been greatly hindered due the difficulty of delivering RNA across biological barriers. While some structure-specific RNA binders through phenotypic screening approaches have been discovered, these usually have been limited by poor selectivity and toxicity issues that prohibits their use in cell-culture and in vivo. One example of an RNA-based technology involves the use of RNA interference (RNAi) which is an essential, post-transcriptional mechanism capable of degrading or blocking particular RNA sequences. This process is triggered when one strand of short, non-coding, double-stranded (ds) RNAs such as endogenous microRNA (miRNA) or synthetic, short interfering RNA (siRNA) is incorporated into the RNA-induced silencing complex (RISC). Once loaded into RISC, these RNAs guide the complex e.g. to complementary messenger RNA (mRNA) sequences which are then targeted for degradation or temporarily stalled in the process of translation.

Offering a specific and efficient means to suppress virtually any target gene, RNAi has become an indispensable research tool and has attracted significant interest as a therapeutic strategy. Despite considerable efforts, however, the widespread application of siRNA-based therapies has been limited due to a lack of effective intracellular delivery methods. As is the case for most oligonucleotide therapeutics, siRNA poorly crosses cellular barriers owing to their size (21-23 base pairs) and negatively-charged character. Moreover, siRNA is easily degraded by ribonucleases (RNases) and has been known to trigger immunogenic responses.

These undesirable characteristics have fueled efforts to develop delivery systems which disguise siRNA and facilitate its translocation and presentation to the RNAi machinery. Diverse siRNA delivery systems have therefore been explored including liposomal nanoparticles, DNA nanotechnology, viral capsid assemblies and sugar- or polymer-derived conjugates.

Accordingly, there remains the need for tuneable carrier systems which can facilitate RNA delivery, in particular delivery of as endogenous microRNA (miRNA) or synthetic, short interfering RNA (siRNA), in a controlled manner, and with a low toxicity.

Also, due to the low stability of RNAs in biological systems, applications for instance involve the need of very high amounts of RNA in order to be effective. Also, RNA may easily break down during delivery to the target cells. Hence, there is a recognized need for specialized constructs designed for the delivery of RNA in particular in a double-stranded form (e.g. for RNAi).

While there are various methods available for directly and indirectly introducing dsRNA into cells, as disclosed for instance in WO2017053720A1, WO2022034946A1, Y. Choi et al., Biomaterials, 35 (2014), 7121-7132, and E. Park et al., Acta Biomaterialia 10 (2014), 4778-4786, it is clear that these methods are generally inefficient, and/or have practical limitations.

Therefore, in view of the foregoing, there exists a need to develop tools and methods for the more efficient delivery of dsRNA into target cells e.g., for the purpose of achieving RNAi.

The present invention aims to provide improved methods and constructs useful in the delivery of dsRNA into eukaryotic target cells. An objective of the present invention is therefore to provide dsRNA constructs with improved penetration properties and enhanced stabilization to be effectively taken up in the target cells.

### BRIEF SUMMARY OF THE DISCLOSURE

Applicants have found that a synthetic multivalent scaffold that can bind to a wide variety of, and dynamic topologies of oligonucleotides, in particular RNA, and is able to deliver RNA compounds into cells, and to release them selectively.

Accordingly, in a first aspect, the present invention relates to a peptide-based compound for complexing and stabilizing a double-stranded oligonucleotide, the compound comprising a structure p-x-b-x′-p′; wherein:
i p and p′ each refer to an oligonucleotide-binding motif;
ii x and x′ each refer to an optional linker motif, and
iii b is a linking motif coupling the oligonucleotide-binding motifs to form a dimerized form,
   wherein motif p and p′ each independently represent a peptide chain having the following fragment comprising a contiguous sequence of at least 14 amino acid residues and having the following general sequence (I), wherein the N-terminal position 1 is located on the left side:

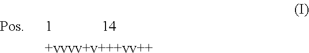

wherein “v” represents a variable amino acid residue position, and wherein “+” represents a position with a positively charged amino acid residue.

Preferably, in the compound according to the present invention motif p and p′ each independently represent a peptide chain having a fragment comprising a contiguous sequence of at most 32 amino acid residues, and having the following general sequence (II), wherein the N-terminal position 1 is located on the left side:

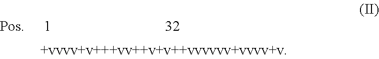

Preferably, in the compound according to the present invention, “v” and “+” represent natural and non-natural amino acids, preferably, wherein “v” represents a variable amino acid residue position and “+” represents Arg, Lys, or His.

Preferably, in the compound according to the present invention, motif p and p′ each independently represent a peptide chain having a fragment comprising a contiguous sequence of at most 32 amino acid residues, and having the following general sequence (III), wherein the N-terminal position 1 is located on the left side:

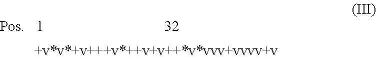

wherein “*” comprises a natural and non-natural polar amino acid residue, in particular.

Preferably, in the compound according to the present invention, each “*” comprises a natural and non-natural amino acid comprising a polar residue, in particular wherein “v” is selected from Glu, Asn or Ser. The present invention further relates to compounds comprising additional side chain-to-side chain crosslinking amino acid residues “#”, and possible combinations thereof, preferably in positions 4, 7, 11, 15, 24, 28 and 32, as applicable.

In a further aspect, the present invention relates to synthetic peptide-based complexation and carrier agent according to the invention that may be modified at one or more site-specific positions with one or more non-natural amino acid residues. These site-specific positions are optimal for substitution of a natural amino acid residue with a non-natural amino acid residue.

In certain embodiments, substitution at these site-specific positions yields oligonucleotide-binding motifs that are uniform in substitution, i.e. that are substantially modified in the selected position. In certain embodiments, a modified peptide substituted at one or more of these site-specific positions has advantageous production yield, advantageous solubility, advantageous binding and/or advantageous activity. The properties of these peptides are described in detail in the sections below.

### BRIEF DESCRIPTION OF THE DRAWINGS

Embodiments of the invention are further described hereinafter with reference to the accompanying drawings, in which:

FIG. 1: (a) shows a crystal structure (PDB ID: 2ZI0) of two TAV2b units (I4-S58 and E5-N64) in complex with double-stranded siRNA. Selected Helix 1 residues involved in RNA-binding (lower left) and dimerization (lower right) are shown in ball-and-stick representation. (b) shows the sequence of TAV2b's Helix 1 (I4-G37) and short peptides used in this study; wherein β=beta-alanine, X=3-mercaptopropionic acid, S5=(S)-2-(4 pentenyl)alanine.

FIG. 2: (a) EMSA of miR-21 in the presence and absence of peptide 1, dimeric peptide 1^{∘∘}1, peptide 2, dimeric peptide 2^{∘∘}2, 3 and control peptide not comprising a dimerization motif. Experiments employed 15% native polyacrylamide gel electrophoresis (PAGE) (c(RNA)=3 μM, c(peptide)=6 μM. Running buffer: 1×TAE, stain: SYBR gold. Cartoon representations of the proposed peptide/RNA complexes corresponding to band species are presented on the right-hand side. (b) Melting temperature profiles of miR-21 in the presence and absence of 1 and 1^{∘∘}1. (c) Melting temperature profiles of miR-21 in the presence and absence of 2 and 2^{∘∘}2.

FIG. 3: (a) CD spectra of miR-21 (c(duplex)=2 μM), 1^{∘∘}1 (c=2 μM), spectra of miR-21 (c(duplex)=2 μM) with dimeric compound 1^{∘∘}1 (c=2 μM) and the sum of the two individual spectra. Buffer: 10 mM sodium phosphate (pH=7.4), 100 mM NaCl. (b) CD spectra of miR21, dimeric compound 2^{∘∘}2, miR-21 with 2^{∘∘}2 and the sum of the two individual spectra.

FIG. 4: (a) Overlaid CD spectra of miR-21 (c(duplex)=2 μMe), 1 (c=2 μM), spectra of miR-21 (c(duplex)=2 μM) with 1 (c=2 μM) and the sum of the two individual spectra (dotted line). Buffer: 10 mM sodium phosphate (pH=7.4), 100 mM NaCl. (b) Overlaid CD spectra of miR-21, 2, miR-21 with 2 and the sum of the two individual spectra (dotted black line).

FIG. 5: (a) Cartoon representation of complex destabilization (unlocking) upon the introduction of excess reducing agent. (b) Table of Tm-values of miR-21 co-incubated dimeric peptide 1^{∘∘}1 and dimeric peptide 2^{∘∘}2 in the absence and presence (red.) of 1 mM TCEP (for melting curves see FIG. 9). (c) EMSA of miR-21 co-incubated with dimeric peptides 1^{∘∘}1 and 2^{∘∘}2 and increasing concentrations of the reducing agent, TCEP. Experiments employed 15% native polyacrylamide gel electrophoresis (PAGE) (c(RNA)=3 μM, c(ligand)=6 μM, c(TCEP)=6, 60 and 600 μM. Running buffer: 1×TAE, stain: SYBR gold.

FIG. 6: (a) CD spectra of miR-21 (c(duplex)=2 μM) co-incubated with (a) 1^{∘∘}1 (c(duplex)=2 μM) in the absence of TCEP (B, buffer: 10 mM sodium phosphate, pH=7.4, 100 mM NaCl), the presence of TCEP (A, reducing buffer: 10 mM sodium phosphate, pH=7.4, 100 mM NaCl, 1 mM TCEP) and the differential spectra of A and B (subtracted spectra A-B, dotted line). (b) Analogous measurements performed with 2002.

FIG. 7: (a) Sequence of Cy5-siRNA (upper) and legend indicating the Cy5-siRNA complex used to treat HEK cells in the proceeding micrograph panels (lower). Confocal micrographs of HEK293 cells after incubation with 1 μM of Cy5-siRNA (b), a solution of Cy5-siRNA and 1^{∘∘}1 (c), a solution of Cy5-siRNA and 2^{∘∘}2 (d), a solution of Cy5-siRNA and 1^{∘∘}1 pre-treated with the reducing agent DTT (e) and a solution of Cy5-siRNA and 2^{∘∘}2 pre-treated with DTT (f).

FIG. 8: (a) Sequences of RNA hairpins (HP 1-5) which bear the same loop (GAUCAA). (b) EMSA of hairpin sequences (HP 1-5, c=1 μM) incubated with 2^{∘∘}2 (c=4 μM). Experiments employed 15% native polyacrylamide gel electrophoresis (Running buffer: 1×TAE). Gel imaged after SYBR™ gold staining.

FIG. 9: Melting temperature profiles of miR-21 in the presence of peptides 1^{∘∘}1 and 2^{∘∘}2 in the presence and absence of TCEP (λ=267 nm, c(miR-21)=2 μM, c(peptide)=2 μM, non-reducing buffer: 10 mM sodium phosphate, pH=7.4, 100 mM NaCl, reducing buffer: 10 mM sodium phosphate, pH=7.4, 100 mM NaCl, 1 mM TCEP. (a) 1^{∘∘}1 in the absence of TCEP and 1^{∘∘}1 in the presence of TCEP, (b) 2^{∘∘}2 in the absence of TCEP and 2^{∘∘}2 in the presence of TCEP.

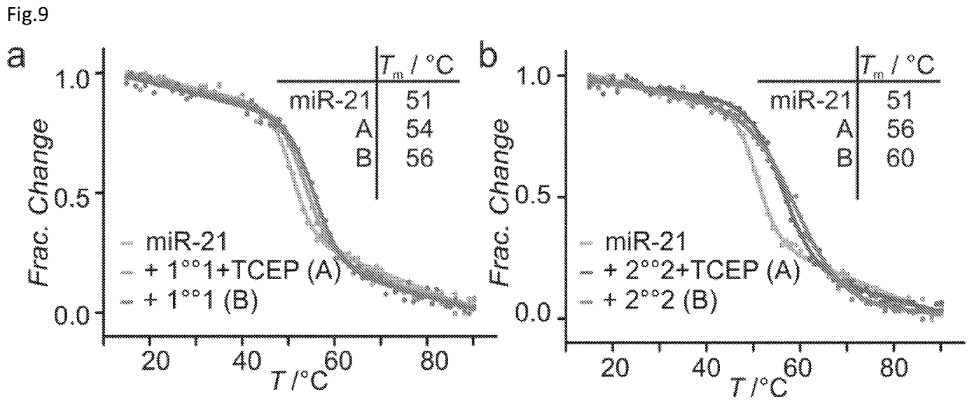

FIG. 10: HPLC chromatograms (λ=210 nm) including peak retention time and corresponding mass spectra of peptides wt33, 1 and 1^{∘∘}1.

FIG. 11: HPLC chromatograms (λ=210 nm) including peak retention time and corresponding mass spectra of peptides 2, 2^{∘∘}2 and 3.

FIG. 12: Binding of peptides to miR-21 as assessed by isothermal titration calorimetry (ITC). (a) peptide 2^{∘∘}2 with miR-21 (concentration (2^{∘∘}2)=305 μM, concentration (miR-21)=15 μM) at 30° C. (b) peptide wt33 with miR-21 (concentration (wt33)=86 μM and concentration (miR-21)=6 μM). (c) peptide 3 with miR-21 (concentration (3)=300 μM and concentration (miR-21)=15 μM).

FIG. 13: Results of the disulfide cleavage assay with a) the chromatograms at λ=200 nm for peptide 2^{∘∘}2 with only peptide (top) and with peptide/RNA complex (bottom), showing complete reduction of the disulfide bond within 5 min, and b) the resulting mass spectra for the reduced peptide 2 (top) and the parental peptide 2^{∘∘}2 (bottom). Calculated m/z of peptide 2: 1257.2 [M+2H]²⁺, 838.5 [M+3H]³⁺, 629.1 [M+4H]⁴⁺, 503.5 [M+5H]⁵⁺. Calculated m/z of peptide 2^{∘∘}2: 1256.5 [M+4H]⁴⁺, 1005.6 [M+5H]⁵⁺, 838.2 [M+6H]⁶⁺, 718.7 [M+7H]⁷⁺, 628.9 [M+8H]⁸⁺, 559.0 [M+9H]⁹⁺.

FIG. 14: Stability of miR-21 in the presence and absence of peptides. Native PAGE of phenol/chloroform extracted miR-21 (concentration=9 μM) after incubation with increasing amounts of FBS for 10 min at 37° C. in presence of a) peptide wt33 (concentration=18 μM) and b) peptide 3 (concentration=18 μM), and c) in presence and absence of peptide 2^{∘∘}2 (concentration=18 μM). Run in TAE, stained with SYBR™ gold. d) 10% native PAGE of phenol/chloroform extracted miR-21 (concentration=9 μM) after incubation with 27.5% fetal bovine serum (FBS) for incremental time points at room temperature in presence and absence of 2^{∘∘}2 (concentration=18 μM).

FIG. 15: Synthesis route of 2^{∘∘}2. Starting with H-Rink Amide Resin, the respective amino acid sequence is synthesized through Fmoc-based solid-phase peptide synthesis (SPPS). Hydrocarbon staple residues (O) get crosslinked by on resin Ring-Closing Metathesis (RCM) followed by coupling of the linker motive 3-mercaptopropionic acid. Final dimerization of two peptides 2 is performed in overnight reaction forming the 2^{∘∘}2 dimer.

### DETAILED DESCRIPTION OF THE INVENTION

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

RNA interference occurs when an organism recognizes double-stranded RNA molecules and hydrolyzes them. The resulting hydrolysis products comprise small RNA fragments of 19-24 nucleotides in length, called small interfering RNAs (siRNAs) or microRNAs (miRNAs). The siRNAs then diffuse or are carried throughout the organism, including across cellular membranes, where they hybridize to mRNAs, or other RNAs, and cause hydrolysis of the RNA.

Interfering RNAs are recognized by the RNA interference silencing complex (RISC) into which an effector strand, or “guide strand” of the RNA is loaded. This guide strand acts as a template for the recognition and destruction of the duplex sequences. This process is repeated each time the siRNA hybridizes to its complementary-RNA target, effectively preventing those mRNAs from being translated, and thus “silencing” the expression of specific genes. In other instances, interfering RNAs may bind to target RNA molecules having imperfect complementarity, causing translational repression without mRNA degradation. The majority of the animal miRNAs studied so far appear to function in this manner.

The term “RNA” includes any molecule comprising at least one ribonucleotide residue, including those possessing one or more natural ribonucleotides of the following bases: adenine, cytosine, guanine, and uracil; abbreviated A, C, G, and U, respectively, modified ribonucleotides, and non-ribonucleotides. “Ribonucleotide” means a nucleotide with a hydroxyl group at the 2′ position of the D-ribofuranose moiety.

As used herein, the terms and phrases “RNA,” “RNA molecule(s),” and “RNA sequence(s),” are used interchangeably to refer to RNA that mediates RNA interference. These terms and phrases include single-stranded RNA, double-stranded RNA, isolated RNA, partially purified RNA, essentially pure RNA, synthetic RNA, recombinant RNA, intracellular RNA, and RNA that differs from naturally occurring RNA by the addition, deletion, substitution, and/or alteration of one or more nucleotides. “mRNA” refers to messenger RNA, which is RNA produced by transcription.

An “interfering RNA” (e.g., siRNA and miRNA) is a RNA molecule capable of post-transcriptional gene silencing or suppression, RNA silencing, and/or decreasing gene expression. Interfering RNAs affect sequence-specific, post-transcriptional gene silencing in animals and plants by base pairing to the mRNA sequence of a target nucleic acid. Thus, the siRNA is at least partially complementary to the silenced gene. The partially complementary siRNA may include one or more mismatches, bulges, internal loops, and/or non-Watson-Crick base pairs (i.e., G-U wobble base pairs).

The terms “silencing” and “suppression” are used interchangeably to generally describe substantial and measurable reductions of the amount of mRNA available in the cell for binding and decoding by ribosomes. The transcribed RNA can be in the sense orientation to effect what is referred to as co-suppression, in the anti-sense orientation to effect what is referred to as anti-sense suppression, or in both orientations producing a double-stranded RNA to effect what is referred to as RNA interference. A “silenced” gene refers to a gene that is subject to silencing or suppression of the mRNA encoded by the gene.

The descriptions “small interfering RNA” and “siRNA” are used interchangeably herein to describe a synthetic or non-natural interfering RNA. The terms “miRNA” or “microRNA” generally refer to natural or endogenous interfering RNAs. As used herein, “miRNA” refers to interfering RNAs that have been or will be processed in vitro or in vivo from a pre-microRNA precursor to form the active interfering RNA. Both siRNAs and miRNAs are RNA molecules of about 19-24 nucleotides, although shorter or longer siRNAs/miRNAs, e.g., between 18 and 26 nucleotides in length, may also be useful. siRNAs or miRNAs may be single stranded or double stranded.

MicroRNAs (miRNAs) are encoded by genes that are transcribed but not translated into protein (non-coding DNA), although some miRNAs are encoded by sequences that overlap protein-coding genes. miRNAs are processed from primary transcripts known as pri-miRNAs to short stem-loop structures called pre-miRNAs that are further processed creating functional siRNAs/miRNAs. Typically, a portion of the precursor miRNA is cleaved to produce the final miRNA molecule. The stem-loop structures may range from, for example, about 50 to about 80 nucleotides, or about 60 nucleotides to about 70 nucleotides, including the miRNA residues, those pairing to the miRNA, and any intervening segments. The secondary structure of the stem-loop structure is not fully base-paired; mismatches, bulges, internal loops, non-Watson-Crick base pairs (i.e., G-U wobble base pairs), and other features are frequently observed in pre-miRNAs and such characteristics are thought to be important for processing. Mature miRNA molecules are partially complementary to one or more messenger RNA molecules, and they function to regulate gene expression. siRNAs of the invention have structural and functional properties of endogenous miRNAs, such as gene silencing and suppressive functions.

Double-stranded RNA inhibition is based on the introduction of RNA into a living cell to inhibit gene expression of a target gene in that cell. The RNA has a region with double-stranded structure. Double-stranded RNA (dsRNA) has the capability to render genes non-functional in a sequence-specific manner. Once introduced into cells, dsRNA can activate mechanisms that target the degradation of cognate cytoplasmic mRNAs and thus can effectively silence full gene expression at the posttranscriptional level. RNAi has been observed in many cell types of divergent eukaryotes, including protozoa, fungi, plants, invertebrates, and mammals. Once inside the target cell, long dsRNA molecules are cleaved into double-stranded small interfering RNAs (siRNAs) that are of from 21 to 25 base pairs in length by an enzyme with RNase III-like activity. Cleavage into siRNAs is an early step in the RNAi silencing mechanism. Hence, introduction of dsRNA can elicit a gene-specific RNA interference response in a variety of organisms and cell types.

Oligonucleotides that share a sufficient degree of complementarity will hybridize to each other under various hybridization conditions. Consequently, oligonucleotides that share a high degree of complementarity thus form strong stable interactions and will hybridize to each other under suitable hybridization conditions. The present invention also relates to complexation, and stabilization of heteroduplexes of DNA and RNA.

The applicants designed synthetically prepared short helical peptide fragments and surprisingly found that when binding two of those together by a disulfide bridge allows a more stable complexation of double stranded oligonucleotides. The resulting homo-dimeric peptides were found suitable as scaffolds binding to the major groove of a dsRNA molecule, resulting in the compounds according to the present invention. These compounds permit the use of shorter, synthetically much more conveniently accessible peptides than those disclosed in the state of the art, but also permits derivatization and modular assembly through dimerization. In addition, the reductive environment in the cytosol can result in cleavage of the disulfide, monomerization of the peptides and therefore reduced affinity for duplex RNA. It is to be understood that the compounds according to the invention may also be connected via other suitable means known in the art that enable a similar stable complexation, optional cleavage of the dimerization connection in the cytosol, and other similar improved effects as disclosed herein. Hence, the present invention is not limited to a disulfide bridge connecting the peptide fragments.

The terms “identical” or “identity,” in the context of two or more peptide sequences, refer to two or more sequences or sub-sequences of motifs in the sequence that are the same.

Sequences are “substantially identical” if they have a percentage of amino acid residues or nucleotides that are the same (i.e., about 60% identity, optionally about 65%, about 70%, about 75%, about 80%, about 85%, about 90%, or about 95% identity over a specified region), when compared and aligned for maximum correspondence over a comparison window, or designated region as measured using one of the following sequence comparison algorithms or by manual alignment and visual inspection.

Optimal alignment of sequences for comparison can be conducted, including but not limited to, by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.); or by manual alignment and visual inspection (see, e.g., Ausubel et al., Current Protocols in Molecular Biology (1995 supplement)).

The term “amino acid” refers to naturally occurring and non-naturally occurring amino acids, as well as amino acids such as proline, amino acid analogues and amino acid mimetics that function in a manner similar to the naturally occurring amino acids.

“Natural” amino acids herein refer to naturally encoded amino acids, namely the proteinogenic amino acids known to those of skill in the art. They include the 20 common amino acids, namely alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine, and the less common pyrrolysine and selenocysteine. Naturally encoded amino acids include post-translational variants of the 22 naturally occurring amino acids such as prenylated amino acids, isoprenylated amino acids, myrisoylated amino acids, palmitoylated amino acids, N-linked glycosylated amino acids, O-linked glycosylated amino acids, phosphorylated amino acids and acylated amino acids. There are rare

The term “non-natural amino acid” refers to an amino acid that is not a proteinogenic amino acid, or a post-translationally modified variant thereof. In particular, the term refers to an amino acid that is not one of the 20 common amino acids or pyrrolysine or selenocysteine, or post-translationally modified variants thereof.

The “non-natural” amino acid can be any non-natural amino acid known to those of skill in the art. In some embodiments, the non-naturally encoded amino acid comprises a functional group. The functional group can be any functional group known to those of skill in the art. In certain embodiments the functional group is a label, a polar group, a non-polar group or a reactive group.

Reactive groups are particularly advantageous for linking further functional groups to the protein at the site-specific position of the protein chain. In certain embodiments, the reactive group is selected from the group consisting of amino, carboxy, acetyl, hydrazino, hydrazido, semicarbazido, sulfanyl, azido and alkynyl.

Those of skill in the art will recognize that proteins are generally comprised of L-amino acids. However, with non-natural amino acids, the present methods and compositions provide the practitioner with the ability to use L-, D- or racemic non-natural amino acids at the site-specific positions. In certain embodiments, the non-natural amino acids described herein include D-versions of the natural amino acids and racemic versions of the natural amino acids.

In the formulas, the dashed lines indicate bonds that connect to the remainder of the peptide chains of the oligonucleotide binding motif, the linker or the dimerization motif. These non-natural amino acids can be incorporated into peptide chains just as natural amino acids are incorporated into the same peptide chains. In certain embodiments, the non-natural amino acids are incorporated into the peptide chain via amide bonds as indicated in the formulas.

The non-natural amino acids may carry different substituents including any functional group without limitation, so long as the amino acid residue is not identical to a natural amino acid residue. In certain embodiments, the substituent can be a hydrophobic group, a hydrophilic group, a polar group, an acidic group, a basic group, a chelating group, a reactive group, a therapeutic moiety or a labelling moiety.

In some embodiments, the non-naturally encoded amino acids include side chain functional groups that react efficiently and selectively with functional groups not found in the 20 common amino acids, including but not limited to, olefinic, azido, ketone, aldehyde and aminooxy groups. For example, a peptide that includes one or more non-naturally encoded amino acid, for instance to form a cycloaddition product that acts as a stable bracket enhancing a conformation that results in a particularly strong affinity to a nucleotide position, thereby enhancing also complex strength, and provide enhanced thermal and/or chemical stability of the complexed nucleotide.

Useful non-natural amino acids may include α-, β-, γ- or otherwise substituted amino acids. Exemplary non-naturally encoded amino acids that may be suitable for use in the present invention and that are useful for reactions with water soluble polymers include, but are not limited to, those with carbonyl, aminooxy, hydrazine, hydrazide, semicarbazide, azide and alkyne reactive groups. In some embodiments, non-naturally encoded amino acids comprise a saccharide moiety. Examples of such amino acids include N-acetyl-L-glucosaminyl-L-serine, N-acetyl-L-galactosaminyl-L-serine, N-acetyl-L-glucosaminyl-L-threonine, N-acetyl-L-glucosaminyl-L-asparagine and O-mannosaminyl-L-serine. Examples of such amino acids also include examples where the naturally-occurring N- or O-linkage between the amino acid and the saccharide is replaced by a covalent linkage not commonly found in nature-including but not limited to, an alkene, an oxime, a thioether, an amide and the like. Examples of such amino acids also include saccharides that are not commonly found in naturally-occurring proteins such as 2-deoxy-glucose, 2-deoxygalactose and the like.

Many of the non-naturally encoded amino acids provided herein are commercially available. Those that are not commercially available are optionally synthesized as provided herein or using standard methods known to those of skill in the art. For example, unnatural amino acids for use in the present invention optionally comprise substitutions in the amino or carboxyl group. Unnatural amino acids of this type include, but are not limited to, α-hydroxy acids, α-thioacids, α-aminothio-carboxylates, including but not limited to, with side chains corresponding to the common twenty natural amino acids or unnatural side chains. In addition, substitutions at the α-carbon optionally include, but are not limited to, L, D, or α-α-disubstituted amino acids such as D-glutamate, D-alanine, D-methyl-O-tyrosine, aminobutyric acid, and the like. Other structural alternatives include cyclic amino acids, such as proline analogues as well as 3, 4, 6, 7, 8, and 9 membered ring proline analogues, β and γ amino acids such as substituted β-alanine and γ-amino butyric acid.

Many unnatural amino acids are based on natural amino acids, such as tyrosine, glutamine, phenylalanine, and the like, and are suitable for use in the present invention. Tyrosine analogs include, but are not limited to, para-substituted tyrosines, ortho-substituted tyrosines, and meta substituted tyrosines, where the substituted tyrosine comprises, including but not limited to, a keto group (including but not limited to, an acetyl group), a benzoyl group, an amino group, a hydrazine, an hydroxyamine, a thiol group, a carboxy group, an isopropyl group, a methyl group, a C₆-C₂₀ straight chain or branched hydrocarbon, a saturated or unsaturated hydrocarbon, an O-methyl group, a polyether group, a nitro group, an alkynyl group or the like. In addition, multiply substituted aryl rings are also contemplated. Glutamine analogues that may be suitable for use in the present invention include, but are not limited to, α-hydroxy derivatives, γ-substituted derivatives, cyclic derivatives, and amide substituted glutamine derivatives. Phenylalanine analogues that may be suitable for use in the present invention include, but are not limited to, para-substituted phenylalanines, ortho-substituted phenyalanines, and meta-substituted phenylalanines, where the substituent comprises, including but not limited to, a hydroxy group, a methoxy group, a methyl group, an allyl group, an aldehyde, an azido, an iodo, a bromo, a keto group (including but not limited to, an acetyl group), a benzoyl, an alkynyl group, or the like. Specific examples of unnatural amino acids that may be suitable for use in the present invention include, but are not limited to, a p-acetyl-L-phenylalanine, an O-methyl-L-tyrosine, an L-3-(2-naphthyl)alanine, a 3-methyl-phenylalanine, an O-4-allyl-L-tyrosine, a 4-propyl-L-tyrosine, a tri-O-acetyl-GlcNAcβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, an isopropyl-L-phenylalanine, and a p-propargyloxy-phenylalanine, and the like. Examples of structures of a variety of unnatural amino acids that may be suitable for use in the present invention are provided in, for example, WO 2002/085923 entitled “In vivo incorporation of unnatural amino acids.”.

The cargo compound according to the invention preferably comprises a biologically active oligonucleotide ribonucleic acid (RNA) molecule that is a double stranded oligonucleotide comprising a microRNA (miRNA) molecule, a small interfering RNA (siRNA) molecule, and/or a DNA molecule.

The present invention also relates to a peptide-based compound or oligonucleotide/peptide-based compound complex according to the invention, for use in increasing the stability of the oligonucleotide cargo and the delivery efficiency of an oligonucleotide cargo to a target eukaryotic cell intended for use in cell therapy, genome editing, adoptive cell transfer, and/or regenerative medicine. Preferably, the target eukaryotic cell is selected from animal cells, mammalian cells; preferably human cells, stem cells, primary cells, immune cells, T cells, and/or dendritic cells.

The present invention also relates to an in vitro method for increasing the delivery efficiency of an oligonucleotide cargo compound to a target eukaryotic cell, comprising contacting the target eukaryotic cell with a peptide-based shuttle agent as set out herein above.

The present invention also relates to an in vitro method for increasing the stability of a double-stranded oligonucleotide compound versus a target eukaryotic cell, the method comprising contacting the oligonucleotide compound with a peptide-based agent according to the invention under conditions suitable to form a shuttle-cargo complex, and for allowing the peptide chains to dimerize. Some shuttle-cargo complexes, albeit with monomeric compounds not having a dimerization motif are disclosed in A. Kuepper et al., Nucleic Acids Res. 2021 Dec. 16; 49(22):12622-1263.

Preferably, the peptide-based compound for complexing and stabilizing a double-stranded oligonucleotide according to the invention is contiguous, i.e. a single molecule. Preferably, the structure p-x-b-x′-p′ according to the invention is contiguous, i.e. a single molecule. Preferably, the connections between the motifs p, x, b, x′, and p′ of the structure p-x-b-x′-p′ according to the invention consist of covalent bonds. Preferably, the connections between the motifs p, b, and p′ consist of covalent bonds. Preferably, the connection between the motifs p and b consists of covalent bonds. Preferably, the connection between the motifs b and p′ consists of covalent bonds. Preferably, the connections between the motifs x, b, and x′ consist of covalent bonds. Preferably, the connection between the motifs x and b consists of covalent bonds. Preferably, the connection between the motifs b and x′ consists of covalent bonds. Preferably, the connection between the motifs p and x consists of covalent bonds. Preferably, the connection between the motifs x′ and p′ consists of covalent bonds.

In a preferred aspect of the invention, provided herein are oligonucleotide-binding motifs comprising a peptide chain having at least one non-natural amino acid residue at a position in the peptide chain that is optimally substitutable. The modified peptide can be in a monomer or dimer form, whereby the dimers can be homodimers or heterodimers. The position in the peptide chain that is optimally substitutable is any position in the peptide chain that can provide a substitution with optimal yield, uniformity, solubility, binding and/or activity. The sections below describe in detail the optimally substitutable positions of such peptide chains.

Preferably, in a further aspect, the present invention relates to compounds, wherein the general peptide sequence (II) is selected from IV to VIII:

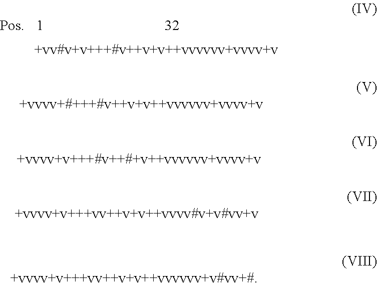

Preferably, the contiguous sequences of p and p′ are selected from general Seq. No 1a (SEQ ID NO 1), which may be varied at positions by additional side chain-to-side chain crosslinking amino acids “#”, and possible combinations thereof, preferably in the position 4, 7, 11, 15, 24, 28 and 32, as shown in Seq. No 1 b (SEQ ID NO 2)) to Seq. No 11 (SEQ ID NO 12)), as set out in Table 1:

TABLE 1

| | | |
|---|---|---|
| KKQAQRKRHKLNRKERGHKSPSEQRRSELWHA | Seq. No 1a | SEQ ID NO 1 |
| KKQ#QRKRHK#NRKERGHKSPSEQRRSELWHA | Seq. No 1b | SEQ ID NO 2 |
| KKQAQR#RHK#NRKERGHKSPSEQRRSELWHA | Seq. No 1c | SEQ ID NO 3 |
| KKQAQRKRHK#NRK#RGHKSPSEQRRSELWHA | Seq. No 1d | SEQ ID NO 4 |
| KKQAQRKRHKLNRKERGHKSPSE#RRS#LWHA | Seq. No 1e | SEQ ID NO 5 |
| KKQAQRKRHKLNRKERGHKSPSEQRRS#LWH# | Seq. No 1f | SEQ ID NO 6 |
| KKQ#QRKRHK#NRKERGHKSPSE#RRS#LWHA | Seq. No 1g | SEQ ID NO 7 |
| KKQ#QRKRHK#NRKERGHKSPSEQRRS#LWH# | Seq. No 1h | SEQ ID NO 8 |
| KKQAQR#RHK#NRKERGHKSPSE#RRS#LWHA | Seq. No 1i | SEQ ID NO 9 |
| KKQAQR#RHK#NRKERGHKSPSEQRRS#LWH# | Seq. No 1k | SEQ ID NO 10 |
| KKQAQRKRHK#NRK#RGHKSPSE#RRS#LWHA | Seq. No 1l | SEQ ID NO 11 |
| KKQAQRKRHK#NRK#RGHKSPSEQRRS#LWH# | Seq. No 1m | SEQ ID NO 12 |

or a sequence comprising at least the first 14 amino acids, counted from the N-Terminus, or a sequence comprising at least the first 15 or 16 amino acids, counted from the N-Terminus.

Advantageously, a fragment p or p′ comprising two motifs “#”-“#” comprises one or more of the complementary substituents a to g that may form a bracket upon cyclisation:

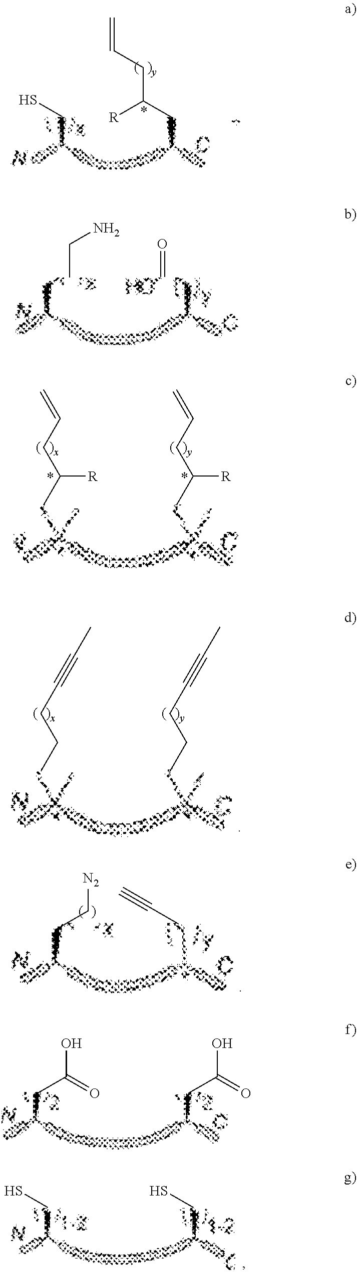

wherein x and y are integers in the range of from 1 to 5, and wherein R represents hydrogen; an optionally substituted C₁-C₆-alkyl; an optionally substituted C₁-C₆-alkenyl; an optionally substituted C₁-C₆ alkynyl.

The present invention also relates to the peptide-based compound, wherein the oligonucleotide-binding motif comprises a cyclic bracket, to enhance its conformational stability. Advantageously, a fragment p or p′ may also comprise two motifs “#”-“#” that have formed a bracket upon cyclisation, whereby the fragment comprises two linked motifs “#”-“#” i to ix, linked either by the cyclisation of a) to e), or from the insertion reaction of f) with vi to form vii, or from the insertion reaction of g) with viii to form ix, respectively:

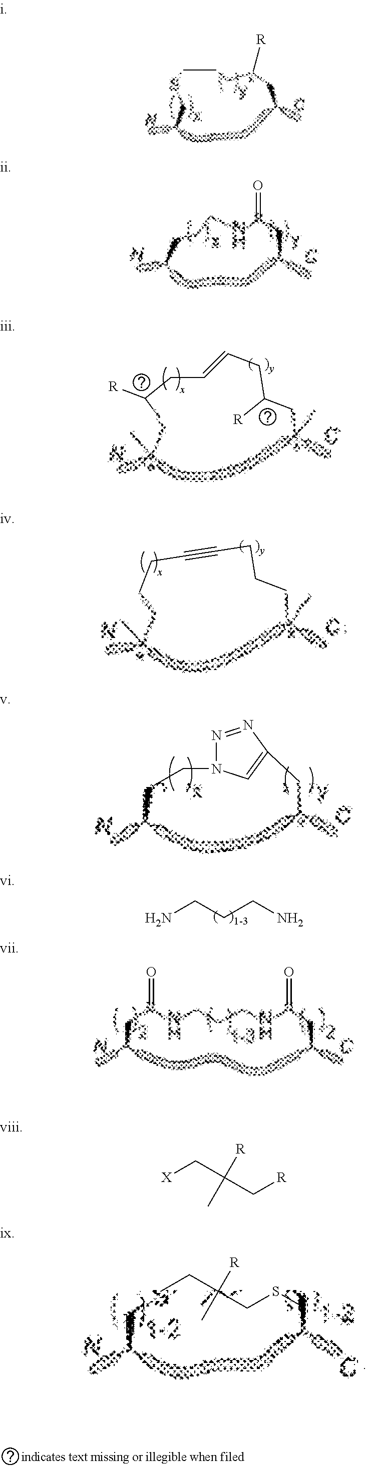

The present invention also relates to a process for the formation of bracket-stabilized compounds, comprising the following reaction schemes:

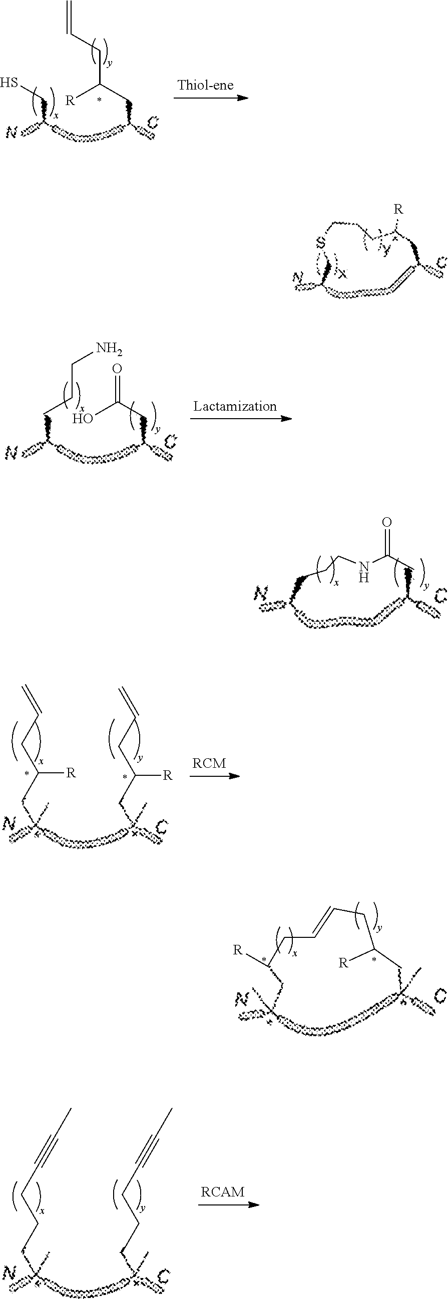

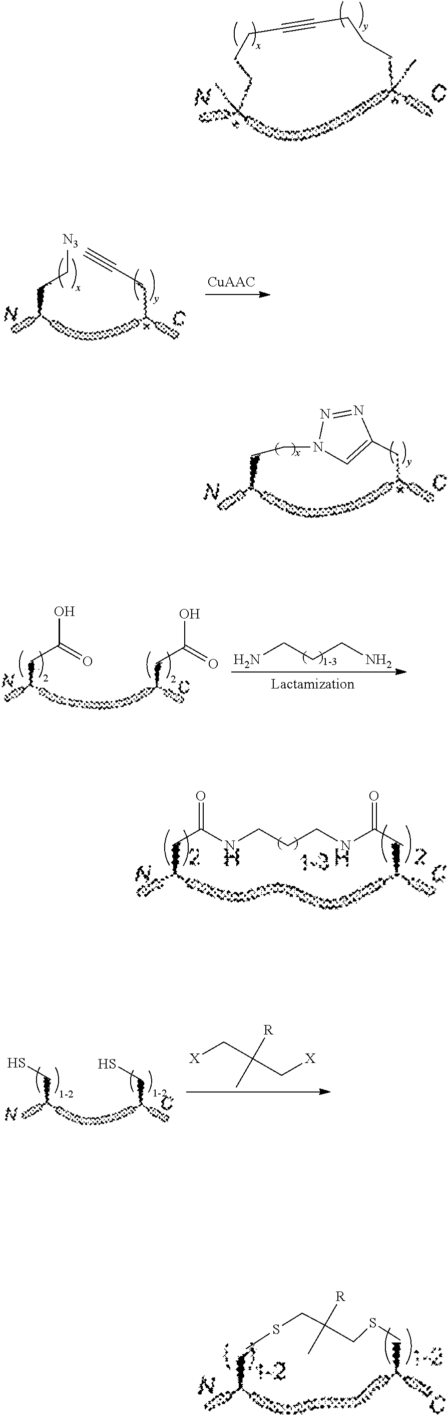

Examples for useful sequences p and/or p′, having side chains are as follows (Table 2):

TABLE 2

| | | |
|---|---|---|
| KKQAQRKRHKLNRKER | Seq. No 1n | SEQ ID NO 13 |
| KKQäQRKRHKöNRKER | Seq. No 1o | SEQ ID NO 25 |
| KKQAQRöRHKöNRKER | Seq. No 1p | SEQ ID NO 26 |
| KKQAQRKRHKöNRKöR | Seq. No 1q | SEQ ID NO 27 |
| KKQAQRKRHKLNRKERGHKSPSEöRRSö | Seq. No 1r | SEQ ID NO 28 |
| KKQAQRKRHKLNRKERGHKSPSEQRRS | Seq. No 1s | SEQ ID NO 29 |
| KKQäQRKRHKöNRKERGHKSPSEöRRSö | Seq. No 1t | SEQ ID NO 30 |
| KKQäQRKRHKöNRKERGHKSPSEQRRS | Seq. No 1u | SEQ ID NO 31 |
| KKQAQRöRHKöNRKERGHKSPSEöRRSö | Seq. No 1v | SEQ ID NO 32 |
| KKQAQRöRHKöNRKERGHKSPSEQRRS | Seq. No 1w | SEQ ID NO 33 |
| KKQAQRKRHKöNRKöRGHKSPSEöRRSö | Seq. No 1x | SEQ ID NO 34 |
| KKQAQRKRHKöNRKöRGHKSPSEQRRS | Seq. No 1y | SEQ ID NO 35 |

Herein, a corresponds to

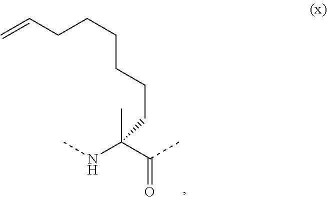

and ö corresponds to

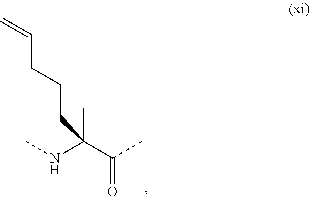

forming the bracket:

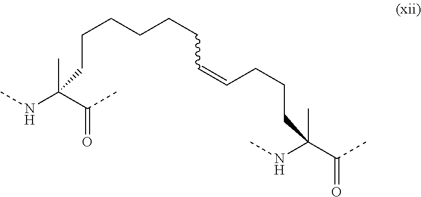

after crosslinking;
or ö and ö correspond to

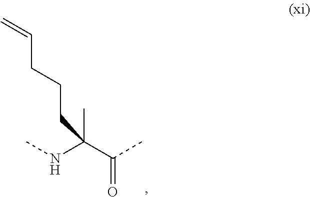

forming a bracket after crosslinking as follows:

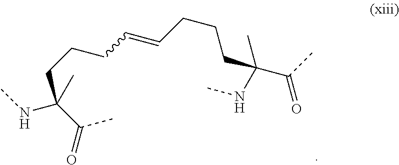

Preferably, any one of the sequences SEQ ID NO: 1, 2, 3, 4, 5, 6, 7, 8, 9, 10, 11, 12, 13, 14, 20, 21, 22, 23, 25, 26, 27, 28, 29, 30, 31, 32, 33, 34, and 35 according to the invention may be a functional variant thereof. Preferably, a sequence comprising at least the first 14 amino acids, counted from the N-Terminus, of any one of the sequences SEQ ID NO: 1, 2, 3, 4, 5, 6, 7, 8, 9, 10, 11, or 12 according to the invention may be a functional variant thereof. Preferably, a sequence comprising at least the first 15 amino acids, counted from the N-Terminus, of any one of the sequences SEQ ID NO: 1, 2, 3, 4, 5, 6, 7, 8, 9, 10, 11, or 12 according to the invention may be a functional variant thereof. Preferably, a sequence comprising at least the first 16 amino acids, counted from the N-Terminus, of any one of the sequences SEQ ID NO: 1, 2, 3, 4, 5, 6, 7, 8, 9, 10, 11, or 12 according to the invention may be a functional variant thereof. Preferably, a sequence comprising at least the first 14, 15, or 16 amino acids, counted from the N-Terminus, of the sequence SEQ ID NO: 1 according to the invention may be a functional variant thereof.

Preferably, the functional variant has at least 70%, 75%, 80%, 85%, 90%, or 95% identity thereof, more preferably at least 90% or 95% identity thereof. Preferably, the functional variant has at least 70%, 75%, 80%, 85%, 90%, or 95% identity, more preferably at least 90% or 95% identity, to the sequence where it is a functional variant of. Such a functional variant can be considered as substantially identical to its parent sequence.

### Dimerization Motif b:

Herein, the term “dimerization motif b” is used interchangeably with the term “linking motif b”. The purpose of motif b is to couple the oligonucleotide-binding motifs to form a dimerized form, hence resulting in motif b also becoming a dimerization motif.

Preferably, in a further aspect, the present invention relates to a compound according to the invention wherein the dimerization motif b comprises a covalent bond, preferably wherein the dimerization motif b consists of one or more covalent bonds. Preferably, according to the invention, the linking motif b coupling the oligonucleotide-binding motifs to form a dimerized form comprises covalently linking or connecting the oligonucleotide-binding motifs, more preferably connecting the N-terminal amino acids of p and p′, or of x and x′. More preferably, the motif b covalently links or covalently connects the oligonucleotide-binding motifs, even more preferably connects the N-terminal amino acids of p and p′, or of x and x′. More preferably, the motif b is a covalent link or covalent connection, even more preferably connecting the N-terminal amino acids of p and p′, or of x and x′. Herein, the term “covalently linking” or “covalently connecting” is understood to mean that a link or connection has been formed via covalent bonds. Preferably, the covalent bond is a covalent bond sensitive to a chemical or physical reaction, for example sensitive to reduction, radiation and/or enzymatic digestion.

Preferably, motif b, together with optional linker motifs x and x′, comprises a structure that enables each of the oligonucleotide-binding motifs to bind to a double-stranded oligonucleotide.

Preferably, in a further aspect, the present invention relates to a compound according to the invention wherein the dimerization motif b comprises a cleavable link. Preferably, the cleavable link comprises a covalent bond sensitive to a chemical or physical reaction, preferably sensitive to reduction, radiation and/or enzymatic digestion. Preferably, motif b comprises a covalent bond sensitive to a chemical or physical reaction, preferably sensitive to reduction, radiation and/or enzymatic digestion. The reactions set out above, including the reaction conditions and catalysts, as applicable, are well known to a skilled artisan.

Preferably, motif b comprises a disulfide bridge, more preferably connecting the N-terminal amino acids of p and p′, or of x and x′.

More preferably, motif b comprises a structure composed of thiol-substituted amino acids covalently bonded through a disulfide bride, according to the general structure (xiv)

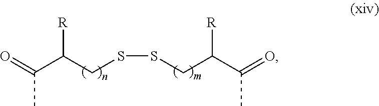

wherein:
n and m each independently represent an integer of from 1 to 4; and
R represents hydrogen; a substituted or unsubstituted alkyl, substituted or unsubstituted alkyl heteroalkyl, a substituted or unsubstituted aryl, —NH₂, —N(H)CH₃COOH, an amide selected from C₂ to C₁₂ aliphatic, optionally alkylated, amidated, or acylated carboxylic acids.

Preferably, in a further aspect, the present invention relates to a compound according to the invention wherein compound according to any one of the preceding claims, wherein the optional linkers x and x′ each independently are selected from polar amino acids, peptides, or —(OCH₂CH₂)_{z}— polyethylene glycol-based linkers, wherein z denotes an integer from 1 to 50.

Preferably, in a further aspect, the present invention relates to a compound according to the invention wherein x and x′ each denotes a peptide according to general formula (xv)

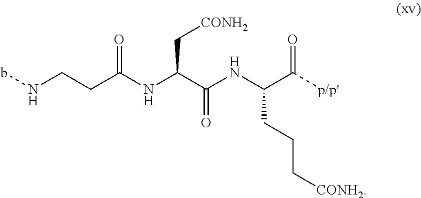

Preferably, in a further aspect, the present invention relates to a compound according to the invention wherein p and p′ are identical, or wherein p and p′ are different. More preferably, the compound is a homodimer.

Preferably, in a further aspect, the present invention relates to a compound according to the invention wherein the motif of p and p′ comprises an amino acids sequence of SEQ ID NO 13 (KKQAQRKRHKLNRKER), wherein motif p and p′ are according to SEQ ID NO 14: (KKQAQRKRHK #NRK #R), and wherein #-# together form a cycle having the general structure (xvi):

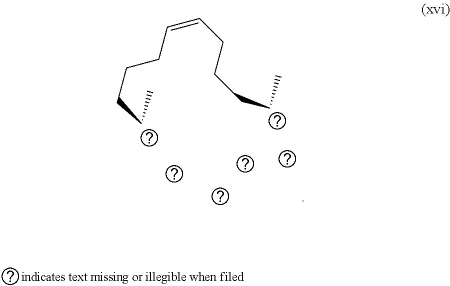

Preferably, in a further aspect, each peptide chain p-comprises a minimum length of at least 14, 15, 16, 17, 18, 19, 20, 21, 22, 23, 24, 25, 26, 27, 28, 29, 30, 31 and up to and including 32 amino acid residues selected from natural or non-natural amino acid residues. Preferably, in a further aspect, each motif p an p′ comprises a helix-forming peptide sequence, wherein the helix-forming peptide sequence comprises at least 50% of positively charged amino acids. Preferably, in a further aspect, each dimerization motif b is convertible into two non-bonded motifs, and wherein the conversion results in reduced affinity of the complex for the double stranded oligonucleotide; preferably, wherein the oligonucleotide is released upon cleavage. Preferably, in a further aspect, each motif p or p′ consists of the amino acid sequence according to SEQ ID NO 13, or a functional variant thereof having at least 85%, 90%, or 95% identity to any one of SEQ ID NO 13.

In a further aspect, the present invention also relates to a monomeric compound for forming a dimeric compound according to the invention or any one of claims 1 to 25, comprising a structure p-x-a; wherein:
i p refers to an oligonucleotide-binding motif;
ii x refers to an optional linker motif, and
iii a is a linkable motif capable of coupling the compound to an identical or different compound to, form a homo- or heterodimer;
   wherein motif p represents a peptide chain having the following fragment comprising a contiguous sequence of at least 14 amino acids, and having the following general sequence (I), wherein the N-terminal position 1 is located on the left side:

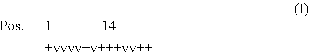

and comprising a contiguous sequence of at most 32 amino acid residues, and having the following general sequence (II):

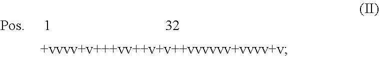

and
preferably, wherein motif p and p′ each independently represent a peptide chain having a fragment comprising a contiguous sequence of at most 32 amino acid residues, and having the following general sequence (III):

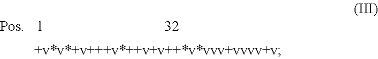

wherein “v” represents a variable amino acid residue position, and wherein “+” represents a position with a positively charged amino acid; wherein “*” comprises a natural and non-natural polar amino acid residue, in particular, wherein “*” is selected from Glu, Asn or Ser.

Preferably, the monomeric compound for forming a dimeric compound according to the invention or any one of claims 1 to 25 refers herein to the peptide-based compound for complexing and stabilizing a double-stranded oligonucleotide according to the invention.

Preferably, p and p′, or x or x′, as applicable, each comprise a N-terminal β-alanine-linked mercaptopropionic acid residue capable to form a disulfide-bridged peptide-based compound upon exposure to basic and oxidative conditions with a second monomeric compound.

In a further aspect, the present invention also relates to a monomeric compound for forming a dimeric compound according to the invention or any one of claims 1 to 25, comprising a structure p-x-a; wherein:
i p refers to an oligonucleotide-binding motif;
ii x refers to an optional linker motif, and
iii a is a linkable motif capable of coupling the compound to an identical or different compound to, form a homo- or heterodimer;
   wherein motif p represents a peptide chain having the following fragment comprising a contiguous sequence of at least 14 amino acids, and having the following general sequence (I), wherein the N-terminal position 1 is located on the left side:

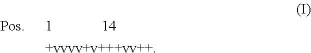

Preferably, the contiguous sequence of p of the monomeric compound according to the invention is selected from general Seq. No 1a (SEQ ID NO 1), which may be varied at positions by additional side chain-to-side chain crosslinking amino acids “#”, and possible combinations thereof, preferably in the position 4, 7, 11, 15, 24, 28 and 32, as shown in Seq. No 1b (SEQ ID NO 2)) to Seq. No 11 (SEQ ID NO 12)), as set out in Table 1:

TABLE 1

| | | |
|---|---|---|
| KKQAQRKRHKLNRKERGHKSPSEQRRSELWHA | Seq. No 1a | SEQ ID NO 1 |
| KKQ#QRKRHK#NRKERGHKSPSEQRRSELWHA | Seq. No 1b | SEQ ID NO 2 |
| KKQAQR#RHK#NRKERGHKSPSEQRRSELWHA | Seq. No 1c | SEQ ID NO 3 |
| KKQAQRKRHK#NRK#RGHKSPSEQRRSELWHA | Seq. No 1d | SEQ ID NO 4 |
| KKQAQRKRHKLNRKERGHKSPSE#RRS#LWHA | Seq. No 1e | SEQ ID NO 5 |
| KKQAQRKRHKLNRKERGHKSPSEQRRS#LWH# | Seq. No 1f | SEQ ID NO 6 |
| KKQ#QRKRHK#NRKERGHKSPSE#RRS#LWHA | Seq. No 1g | SEQ ID NO 7 |
| KKQ#QRKRHK#NRKERGHKSPSEQRRS#LWH# | Seq. No 1h | SEQ ID NO 8 |
| KKQAQR#RHK#NRKERGHKSPSE#RRS#LWHA | Seq. No 1i | SEQ ID NO 9 |
| KKQAQR#RHK#NRKERGHKSPSEQRRS#LWH# | Seq. No 1k | SEQ ID NO 10 |
| KKQAQRKRHK#NRK#RGHKSPSE#RRS#LWHA | Seq. No 1l | SEQ ID NO 11 |
| KKQAQRKRHK#NRK#RGHKSPSEQRRS#LWH# | Seq. No 1m | SEQ ID NO 12 |

or a sequence comprising at least the first 14 amino acids, counted from the N-Terminus, or a sequence comprising at least the first 15 or 16 amino acids, counted from the N-Terminus.

In a further aspect, the present invention also relates to a complex comprising a compound according to the invention, further comprising a double-stranded oligonucleotide, preferably an siRNA or a hairpin RNAi compound.

In a further aspect, the present invention also relates to peptide-based compound or oligonucleotide/peptide-based compound complex, for use as a shuttle and release agent to facilitate delivery of the complexed oligonucleotide to a target eukaryotic cell, and preferably for releasing the oligonucleotide cargo into the cell by modulation of the oligonucleotide binding in situ, and/or for the stabilization of the oligonucleotide.

In a further aspect, the present invention also relates to peptide-based compound or oligonucleotide/peptide-based compound complex, for use in a clinical or therapeutic in vivo method for increasing the transduction efficiency of the oligonucleotide into the target eukaryotic cell, wherein the cargo is a biologically active oligonucleotide, preferably for use in cell therapy, genome editing, adoptive cell transfer, and/or regenerative medicine.

Preferably, the compound or the oligonucleotide-peptide complex are employed at a concentration sufficient to increase the transduction efficiency of the cargo compound to the target eukaryotic cell. Preferably, the biologically active oligonucleotide ribonucleic acid (RNA) molecule is a double stranded oligonucleotide comprising a microRNA (miRNA) molecule, a small interfering RNA (siRNA) molecule, and/or an RNA/DNA molecule. Preferably, the target eukaryotic cell is selected from animal cells, mammalian cells; preferably human cells, stem cells, primary cells, immune cells, T cells, and/or dendritic cells.

In a further aspect, the present invention also relates to an in vitro method for increasing the transduction efficiency of an oligonucleotide cargo compound to the target eukaryotic cell, comprising contacting the target eukaryotic cell with a compound according to the present invention.

In a further aspect, the present invention also relates to an in vitro method for increasing the stability of a double stranded oligonucleotide compound, the method comprising contacting the oligonucleotide compound with the peptide-based agent under conditions suitable to form a shuttle-cargo complex, and for allowing the peptide chains to dimerize.

In a further aspect, the present invention also relates to a compound, having the general structure xvii:

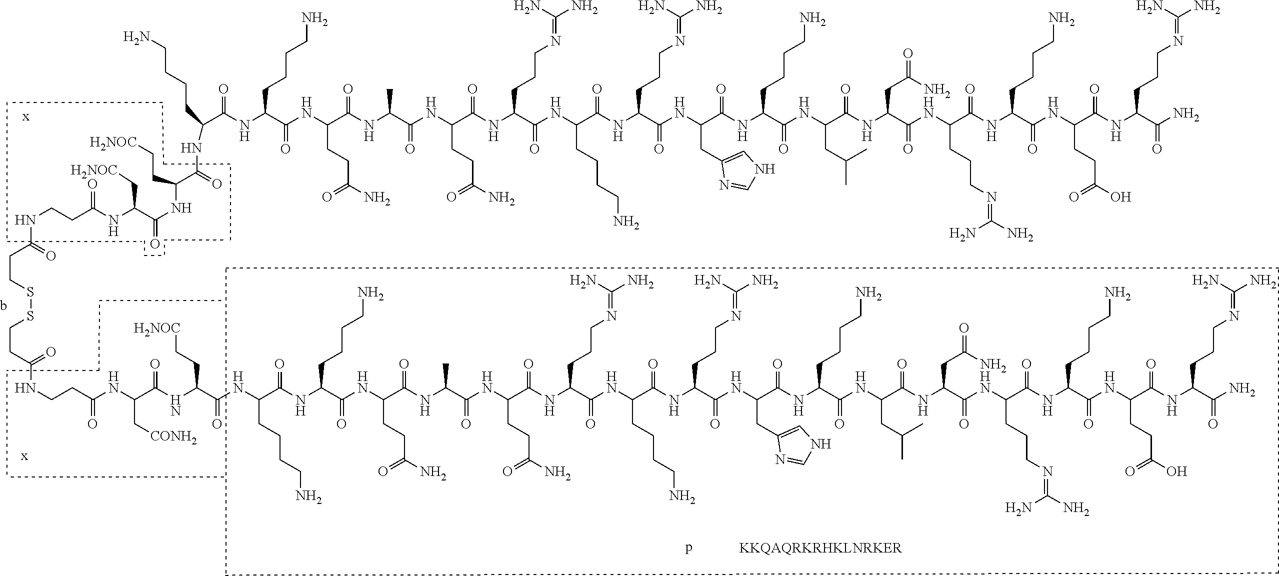

(xvii).

In a further aspect, the present invention also relates to a compound, having the general structure xviii:

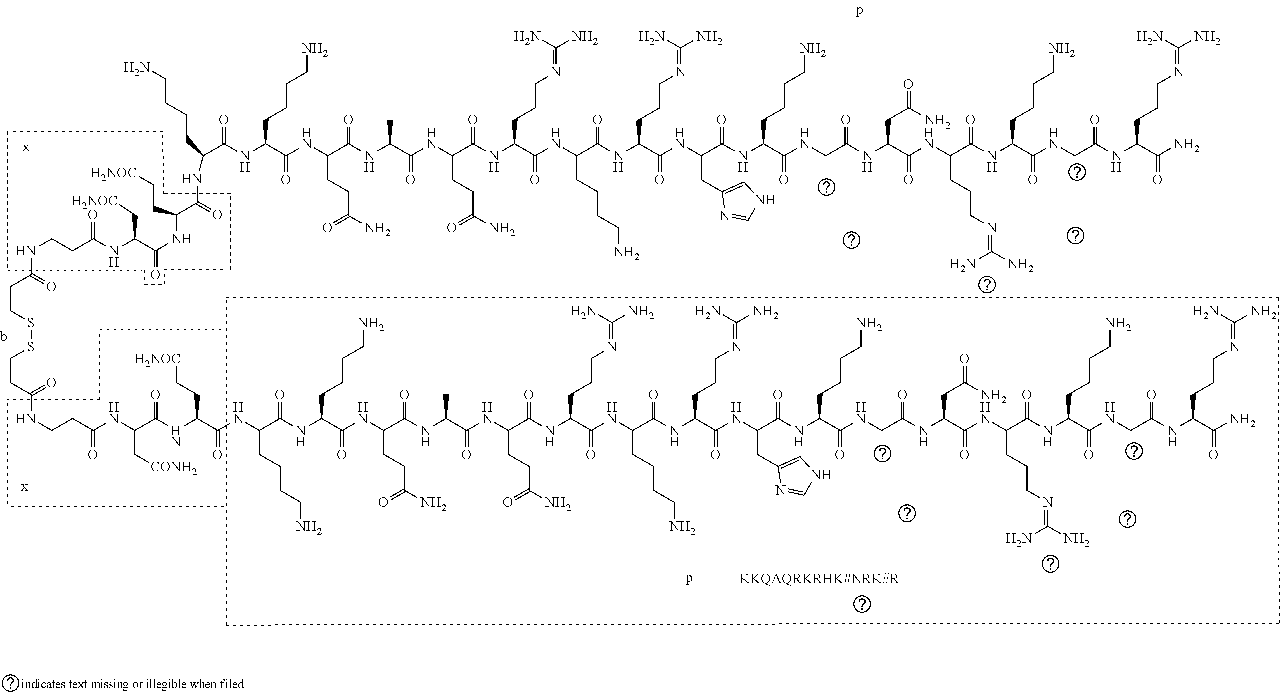

(xviii).

Applicants found that such peptide-based agents binding double-stranded RNA can be designed and synthesized by making use of motifs that carry a significant amount of positively charged amino acids; and by adding a dimerization motif which converts the peptide into a reversible homodimer. The dimer can bind dsRNA to form a compact delivery vehicle, which may advantageously be suitable for deep tissue penetration and extension with additional functionalities, e.g. targeting and/or pharmacokinetic life-time enhancement. The cleavable character of the dimeric peptide also allows for an intracellular release of the RNA cargo, thereby reducing the amount of RNA and peptide required.

Such peptide-oligonucleotide complexes exhibited an enhanced stability and cellular permeability. In one example, siRNA was delivered using a helical stapled peptide that underwent disulfide-mediated peptide dimerization. The reductive cleavage of the peptide dimers in a reducing environment was found to lead to disassembly of the oligonucleotide/peptide-based compound complexes, thereby releasing the siRNA cargo after cellular uptake.

In yet another aspect, the present invention relates the agent according to the invention, for use as a shuttle and release agent to shuttle the oligonucleotide/peptide-based compound complexes into a target eukaryotic cell, and to release the oligonucleotide cargo into the cytosol by modulation for dsRNA-binding in situ. In a further aspect, the present invention relates to a peptide-based shuttle agent, for use in a clinical or therapeutic in vivo method for increasing the transduction efficiency of a cargo to a target eukaryotic cell, wherein the cargo is a biologically active oligonucleotide.

In a further aspect, the present invention relates to an in vitro method for increasing the transduction efficiency of an oligonucleotide cargo compound to a target eukaryotic cell, comprising contacting the target eukaryotic cell with a peptide-based shuttle agent.

In yet a further aspect, the present invention relates to an in vitro method for increasing the stability of a double stranded oligonucleotide compound, the method comprising contacting the oligonucleotide compound with a peptide-based agent according to the invention under conditions suitable to form a shuttle-cargo complex, and allowing the peptides to dimerize.

In a further aspect, provide herein are complexes of the peptide with one or more cargo oligonucleotide molecules. The cargo molecule can be any molecule deemed useful for conjugating to a modified protein. In certain embodiments, the cargo molecule can be a therapeutic molecule or a diagnostic molecule. Advantageously, in certain embodiments, the non-natural amino acids of the peptide-based compound provide sites useful for linking to a linker or to the cargo molecule. Accordingly, provided herein are complexes comprising a peptide linked to a cargo moiety through a series of positively charged amino acids, which fit into the groove of the Ds oligonucleotide.

In another aspect, provided herein are methods of making the modified proteins. The peptides can be made by any technique apparent to those of skill in the art for incorporating non-natural amino acids into site-specific positions of protein chains.

Preferably, the peptides are made by solid phase synthesis, but may also be prepared by a semi-synthesis, in vivo translation, in vitro translation or cell-free translation.

In another aspect, provided herein are methods of making the complexes of the compounds. These complexes can be made by any technique apparent to those of skill in the art for incorporating non-natural amino acids into site-specific positions of protein chains and for linking the proteins to payload molecules.

In another aspect, provided herein are methods of using the complexes for therapy. Compounds or complexes directed to a therapeutic target can incorporate one or more site-specific non-natural amino acids according to the description herein. These oligonucleotide/peptide-based compound complexes can be used for treating or preventing a disease or condition associated with the therapeutic target.

Advantageously, a site-specific non-natural amino acid is used to link the protein to a therapeutic payload to facilitate efficacy. Exemplary complexes, therapeutic targets and diseases or conditions are described herein.

In another aspect, provided herein are methods of using the oligonucleotide/peptide-based compound complexes for detection. Complexes can incorporate one or more site-specific non-natural amino acids according to the description herein. The peptide-based compounds can be used with a label to signal binding to the detection target. Advantageously, a site-specific non-natural amino acid can be used to link the modified protein to a label to facilitate detection. Exemplary peptide complexes, detection targets and labels are described herein.

In another aspect, provided herein are methods of modifying the stability of payload molecules. Peptide-based compounds can be modified with a non-natural amino acid as described herein to facilitate binding to a payload molecule thereby modifying the stability of the payload molecule. For instance, a payload molecule can be bound to the peptide-based compound to increase the in vivo stability of the payload molecule. Exemplary payload molecules and linking moieties are described herein.

### EXPERIMENTS

The following, non-limiting experiments illustrate the present invention.

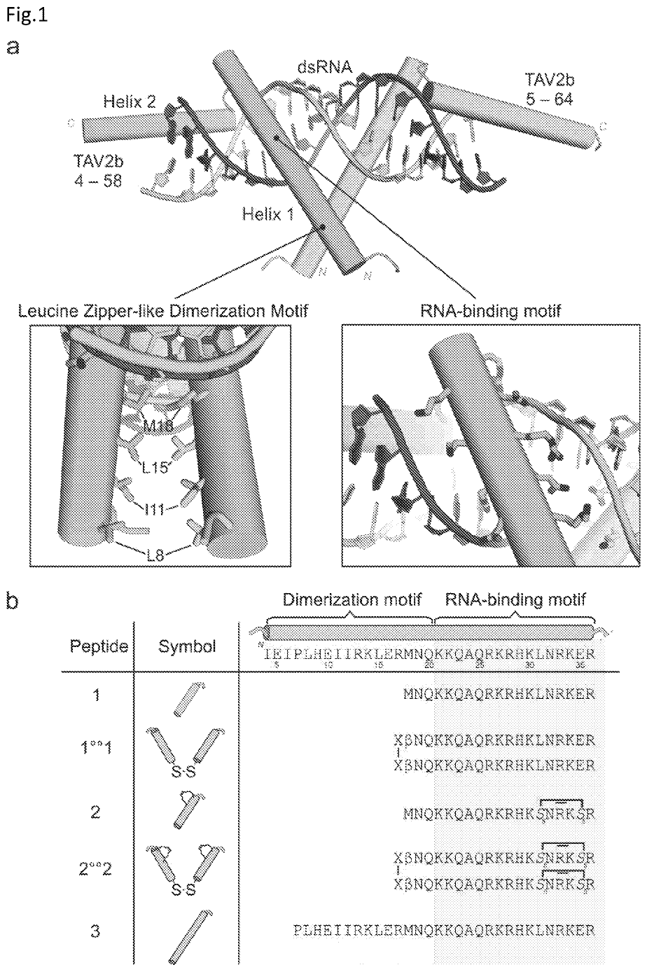

A particularly suitable position for the introduction of a dimerization position, given its proximity to the RNA-binding motif and the short distance between respective peptide monomers with binding motif Sequence 1 was amino acid position M18 (FIG. 1a, lower left). Furthermore, an 18-amino acid fragment extending from M18 to the end of Helix 1's RNA-binding motif (peptide 1, M18-R36) was found as a particularly suitable monomeric scaffold.

As a particularly suitable covalent link between the individual peptide fragments, applicants incorporated an N-terminal disulfide motif. Aside from their synthetic accessibility, it was found that disulfides offer uniquely reversible covalent linkages which are vulnerable to reducing agents, providing a useful point of modulation for dsRNA-binding in situ.

As a means to compare the dsRNA recognition abilities of peptides to a TAV2b leucine zipper-like dimerization motif, an extended peptide fragment (peptide 3, FIG. 1b) was also included in this initial series. Peptides were synthesized according to standard solid-phase synthesis procedures.

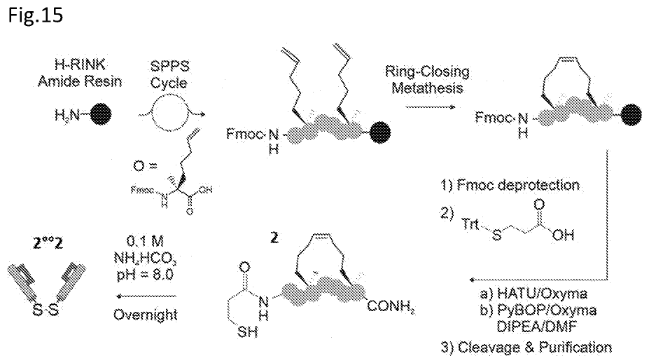

In the case of stapled peptide monomer 2 and dimer 2^{∘∘}2, ring-closing metathesis (RCM) was performed on resin after sequence assembly. Once cleaved, thiol-equipped peptides 1° and 2° were dimerized via overnight incubation in ammonium bicarbonate buffer (pH=8.0). For an overview of the synthesis of peptide monomer 2 and dimer 2^{∘∘}2, see FIG. 15.

Advantageously, to obtain a dimer of peptides, residue M18 was substituted for a β-alanine-linked mercaptopropionic acid moiety (XP, FIG. 1b) which upon incubation in a basic, oxidative buffer system forms a disulfide-bridged peptide (1^{∘∘}1, FIG. 1b).

To stabilize the α-helical conformation of monomer 1 and dimer 1^{∘∘}1, peptides incorporating all-hydrocarbon staples were advantageously also pursued. Based on the sequence of the high affinity, peptides 2 and 2^{∘∘}2 (FIG. 1b) were designed where L31 and E35 are replaced by terminal alkene-baring building blocks which may, through ring-closing olefin metathesis (RCM), be crosslinked to form an inter-side chain macrocycle.

With the desired peptides in hand, we first assessed their dsRNA-binding potential using an electrophoretic mobility shift assay (EMSA). Making use of non-denaturing conditions, EMSA allows to resolve biomolecular complexes by size and charge character. EMSAs allow to examine the binding interaction of dsRNA and wt33, a 33-amino acid peptide which contains the RNA-binding motif of TAV2b's Helix 1 and 2, but does not have a dimerization motif, was compared.

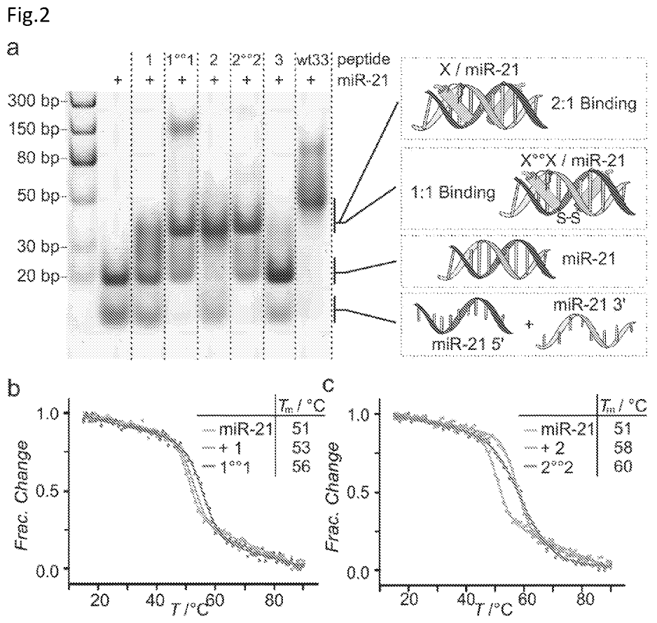

WT33 was employed as comparative positive control for peptide binding while the double-stranded microRNA, miR-21, was chosen as a sample dsRNA target for this assay. Comprised of an 18 base pair (bp) duplex, miR-21 can accommodate the binding of two wt33 monomers, similar to siRNA duplexes. Under established EMSA conditions, miR-21 resolves as two bands, a lower band corresponding to both unbound single strands and a higher, more distinct band (ca. 20 bp) corresponding to the miR-21 duplex (FIG. 2a). Incubation with wt33 leads to formation of a smeared elevated band (ca. 50 bp), corresponding to a 2:1, peptide/dsRNA complex (right, FIG. 2a). Upon coincubation with peptide 1, streaking was observed above the dsRNA band, indicative of a low affinity interaction between 1 and the RNA duplex (FIG. 2a). In contrast, incubating miR-21 with 1^{∘∘}1, leads to the formation of two discrete elevated bands, a reduction in the intensity of the dsRNA band and the disappearance of the ssRNA band (FIG. 2a). With reference to wt33, the most prominent elevated band (ca. 30 bp) likely corresponds to a 1:1 peptide/dsRNA complex while the upper band may represent a higher order structure. The reduction in ds and ssRNA band intensity is likely a result of these binding events. In the case of stapled peptide monomer 2, incubation with miR-21 yields a similar elevated band (ca. 30 bp) indicative of a 2:1 complex (FIG. 2a). A comparable band is also observed for dimer 2^{∘∘}2 however unlike dimer 1^{∘∘}1 no other bands corresponding to marker sizes higher than 50 bp are observed (FIG. 2a). Surprisingly, weak dsRNA binding was observed for peptide 3 (FIG. 2a).

A thermal denaturation assay was next used to further characterize the dsRNA binding abilities of our peptides. Typically employed in the study of nucleic acid complexes, thermal denaturation assays make use of the spectral changes resulting from complex unfolding as temperature is increased. Using circular dichroism (CD) spectroscopy as a readout, we measured the changes in ellipticity at λ=267 nm, the wavelength maxima associated with A-form dsRNA. In line with previous measurements, the mid-point of denaturation or melting temperature (Tm) associated with miR-21 is 51° C. (FIG. 2b and FIG. 2c). To assess the stability of peptide-dsRNA complexes, peptides were co-incubated with miR-21 at an equimolar concentration (c=2 μM) and measured analogously. Likely reflecting its low affinity for miR-21, the addition of peptide 1 yielded only a minor improvement in thermal stability (Tm=53° C., FIG. 2b). Contrastingly, incubation with dimeric peptide 1^{∘∘}1 leads to a greater increase stabilization (Tm=56° C., FIG. 2b) which was also seen for peptide 2 (Tm=58° C., FIG. 2c). Addition of dimeric peptide 2^{∘∘}2 led to the largest increase in thermal stability (Tm=60° C., FIG. 2c), notably exceeding positive control wt33 (Tm=58° C.), indicative of strong complex stability. CD spectroscopy allows not only the measurement of thermal denaturation profiles but can also be used to compare the structural characteristics of biomolecular complexes.

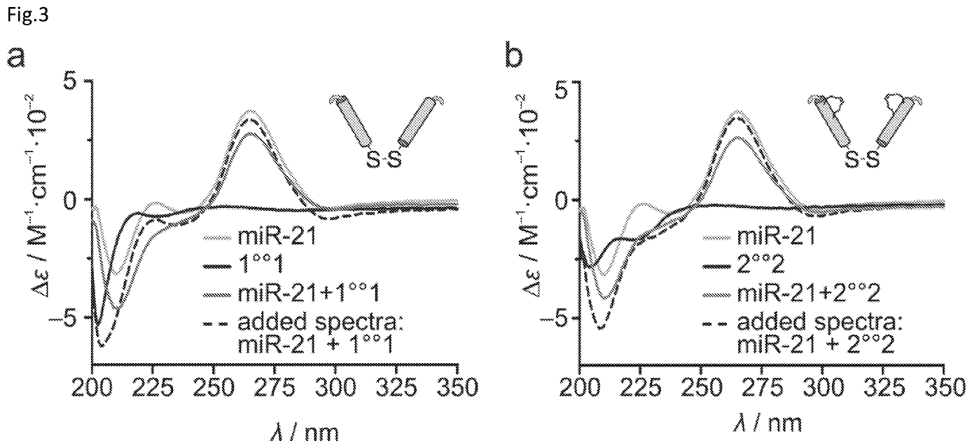

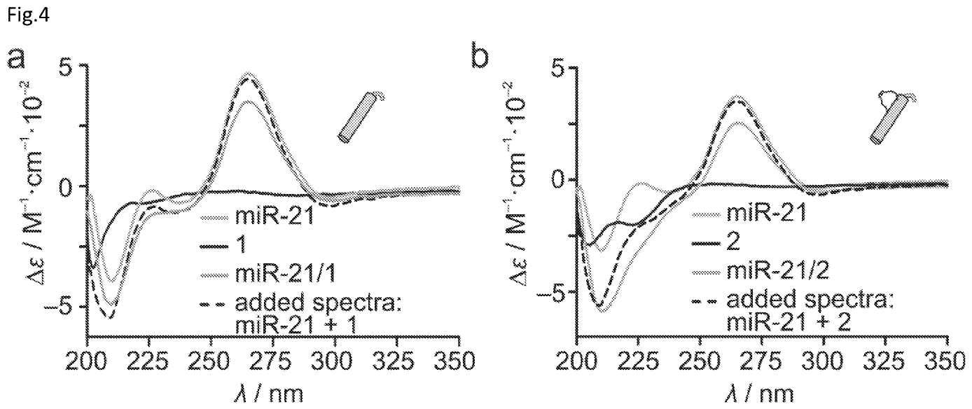

To gain insight into the nature of peptide binding to dsRNA, the CD spectra of peptides 1, dimer compound 1^{∘∘}1, compound 2 and dimer compound 2^{∘∘}2 as well as miR-21 alone and in the presence of each peptide were measured. miR-21 displays a spectra typical of an A-form dsRNA duplex (λ(min)=210 nm, λ(max)=267 nm), while peptides 1 and 1^{∘∘}1 both display spectra corresponding to random coil type structures (FIG. 3a and FIG. 4a). Stapled peptides 2 and 2^{∘∘}2 on the other hand yielded characteristic, alpha-helical spectra (λ(min1)=208 nm, λ(min2)=222 nm, FIG. 3b and FIG. 4b). Likely relating to the distortion of the duplex structure upon peptide binding, co-incubation of miR-21 with each of the peptide was found to lower the observed dsRNA maxima (FIG. 3 and FIG. 4). For both dimers 1^{∘∘}1 and 2^{∘∘}2, co-incubation also led to noticeable changes in ellipticity values in the region between λ=208 and 222 nm (FIG. 3). These changes in ellipticity cannot be accounted for by the simple addition of both spectra (dotted lines, FIG. 3) and likely result from peptide helical induction upon dsRNA binding.

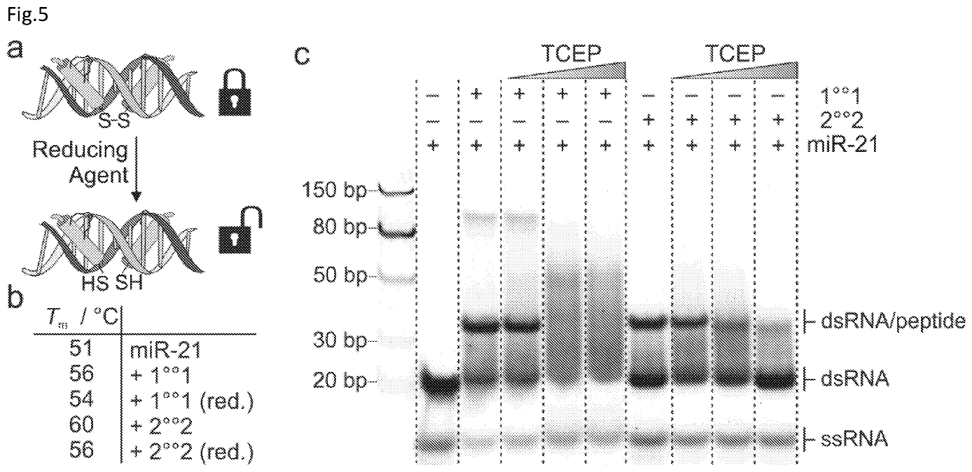

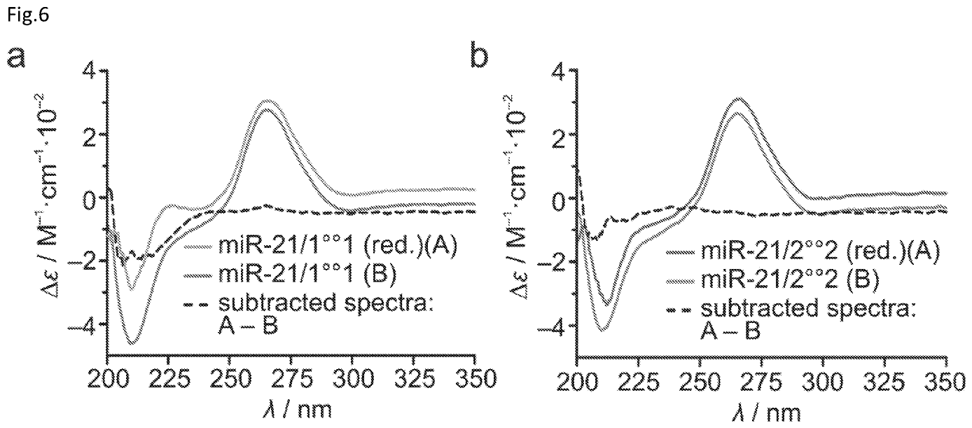

Having observed a favourable impact of disulfide dimerization on peptide affinity for dsRNA, it was sought to probe how disulfide reduction could be used to chemically modulate the stability of dsRNA/peptide complexes. Conceptually, we envisaged that the introduction of excess reducing agent could act to molecularly unlock the dsRNA-shifting the bound dsRNA from a high stability peptide complex to a lower stability structure (FIG. 5a). To assess the possibility of the proposed destabilization approach, first thermal denaturation experiments were performed. Here, treatment with an excess of the reducing agent, tris(2-carboxyethyl)phosphine (TCEP) was found to lower the melting temperatures of dsRNA complexes containing either 1^{∘∘}1 or 2^{∘∘}2 (ΔTm=2° C. and 4° C. respectively, FIG. 5b). In line with this observation, CD spectroscopy revealed that TCEP-treated complexes display reduced ellipticity values in the region around λ=208 and 222 nm (FIG. 6). Such changes in ellipticity point towards a loss in peptide alpha-helical character in the RNA-bound state.

Additionally, EMSA experiments were performed where dsRNA was incubated with either peptide 1^{∘∘}1 or 2^{∘∘}2 in the presence or absence TCEP. As seen previously, coincubation of either dimer with dsRNA led to the formation of a discrete elevated band, indicative of complex formation (FIG. 5c). Introduction of increasing concentrations of TCEP led to a reduction in the intensity of this band and an increase in the intensities of bands associated with both ds- and ssRNA (FIG. 5c), suggesting that disulfide reduction leads to partial disassembly or unlocking of the dsRNA-peptide complex. Having verified the tuneable, dsRNA-binding abilities of our peptide dimers, their use a cellular siRNA delivery tools was confirmed.

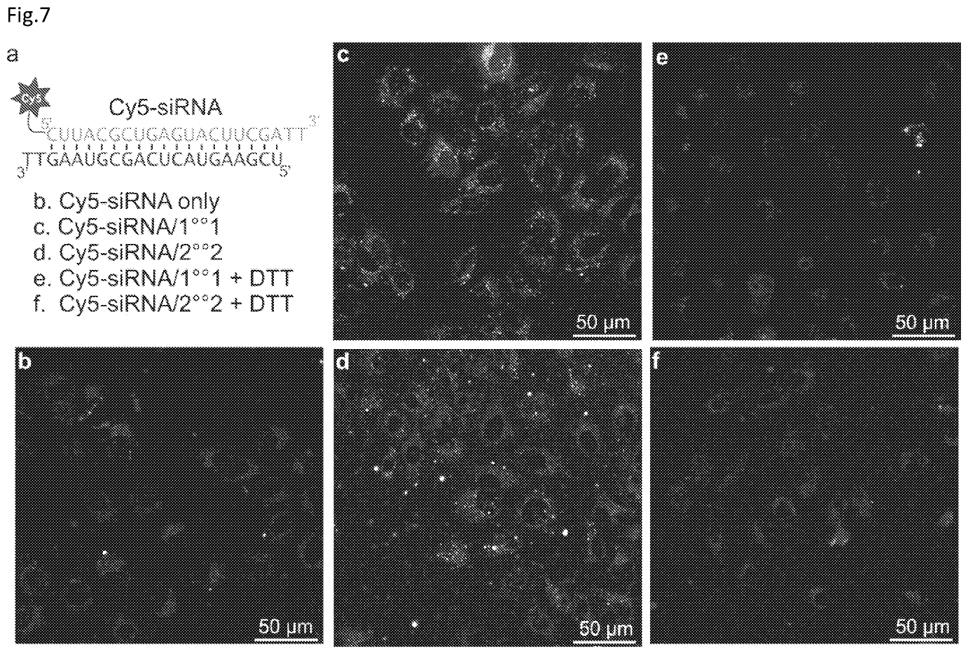

For that purpose, a 21 nt long siRNA comprised of a 19 bp stem and equipped with the far-red fluorescent label, cyanine 5 (Cy5) to monitor RNA internalization (Cy5-siRNA, FIG. 7a) was employed. Initially, HEK293 cells were incubated with Cy5-siRNA (c=1 μM, 37° C., 1 h) before being imaged by confocal fluorescence microscopy. Cy5-siRNA alone showed relatively low cellular uptake (FIG. 7b) which was not improved when co-incubated with either peptide 1 or peptide 2 (c(peptide)=0.5 μM, c(RNA)=1 μM). However, upon coincubation with either peptide 1^{∘∘}1 or 2^{∘∘}2, an increase in fluorescence intensity was observed, indicative of enhanced siRNA internalization (FIG. 7c and FIG. 7d). To assess the tunability of this effect, Cy5-siRNA/1^{∘∘}1 or Cy5-siRNA/2^{∘∘}2 complexes were also pre-treated with the reducing agent dithiothreitol (DTT) in order to promote complex disassembly. Notably, DTT treatment was found to decrease cellular fluorescence intensities for both complexes, yielding micrograph profiles comparable to those observed for Cy5-siRNA alone (FIG. 7e and FIG. 7f).

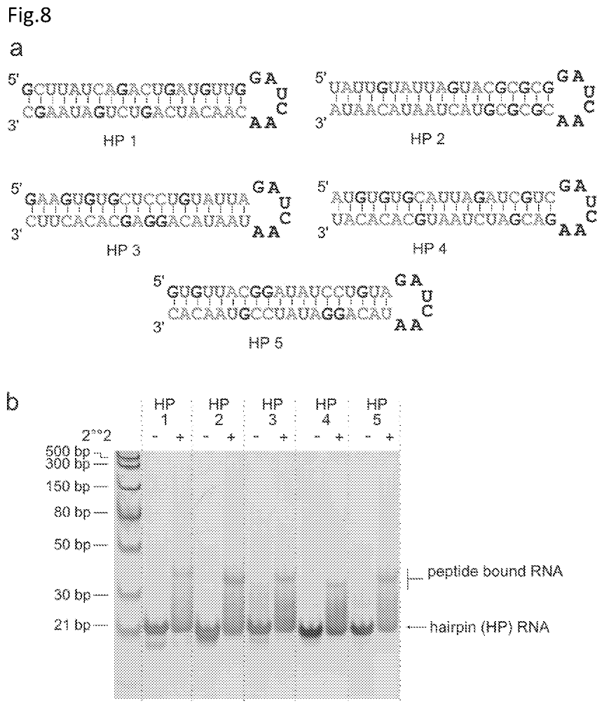

The binding of 2^{∘∘}2 to a set of five ds RNA hairpins, composed of different, complementary 19 base pair stems bridged via a fixed 6 nucleotide loop (FIG. 8a). In EMSA experiments, 2^{∘∘}2 showed binding to each hairpin, resulting in the occurrence of the expected new bands (FIG. 8b) indicating indeed sequence-independent binding.

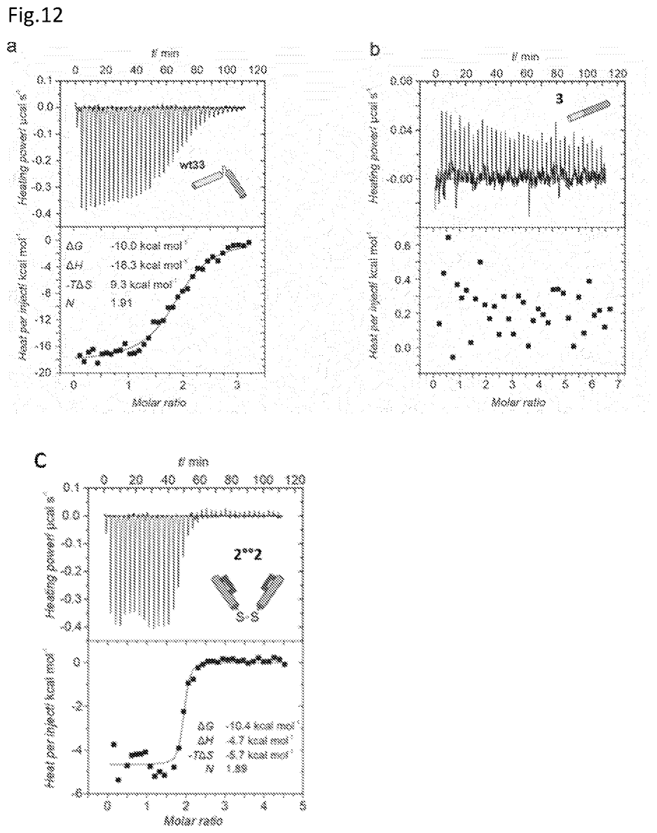

To confirm RNA-binding, isothermal titration calorimetry (ITC) was used. For peptide wt33, the expected good affinity for miR-21 can be observed (Kd=328 nM, FIG. 12a). Interestingly, dimeric peptide 2^{∘∘}2 shows a 10-fold higher affinity (Kd=31.7 nM, FIG. 12c) than wt33 while exhibiting a lower enthalpic contribution to binding (ΔH=4.7 and 18.3 kcal mol⁻¹ for 2^{∘∘}2 and wt33, respectively). However, this lower enthalpic contribution is compensated by a greatly reduced entropic penalty (−TΔS=5.7 and 9.3 kcal mol⁻¹ for 2^{∘∘}2 and wt33, respectively). Notably, for both peptides, an N-value around two (N=1.89 and 1.91 for 2^{∘∘}2 and wt33, respectively) can be observed indicating the binding of two peptides per miR-21 duplex. For wt33 this is in line with applicants' previously reported observations. For monomeric peptide 3 bearing the leucine zipper motive, no binding to miR-21 was detected by ITC (FIG. 12b) confirming the EMSA findings (FIG. 2a).

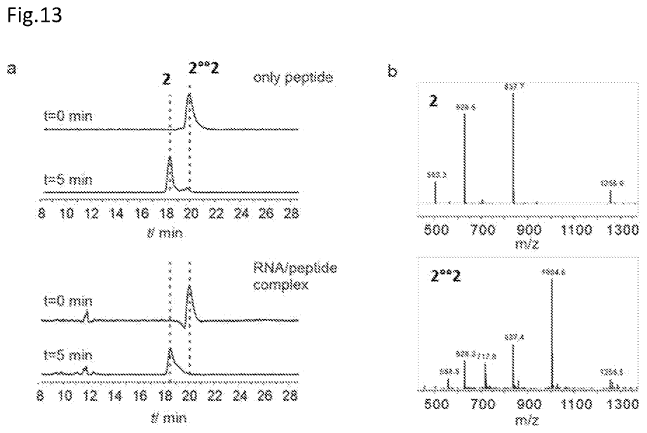

Disulfide cleavage in 2^{∘∘}2 was tested via the Disulfide Cleavage Assay in the presence of intracellular glutathione concentrations (concentration=5 mM). Based on LC-MS, peptide monomerization was observed in less than 5 min both in the presence and the absence of miR 21 (FIG. 13).

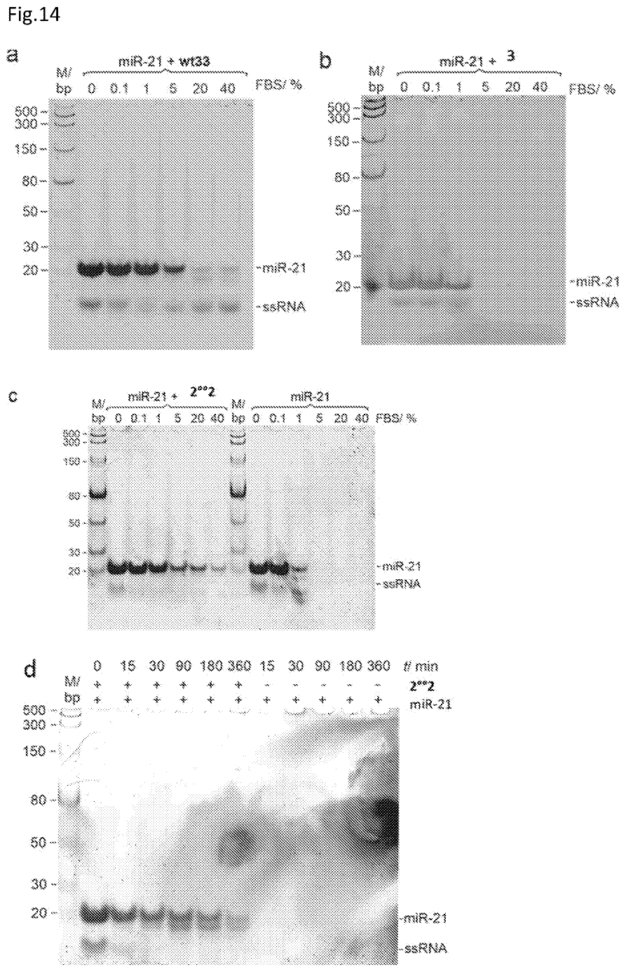

The rapid degradation of RNA by nucleases in serum is one of the central challenges associated with the therapeutic use of RNA. Applicants were therefore interested whether peptide binding promotes RNA stability in serum. For this purpose, miR-21 was incubated with different concentrations of fetal bovine serum (FBS) at 37° C., followed by phenol/chloroform extraction and PAGE analysis (FIG. 14). In the absence of peptide 2^{∘∘}2, miR-21 (concentration=9 μM) exhibited the expected rapid degradation within 10 minutes at 1% FBS and higher concentrations (FIG. 14c, right). Notably, peptide 2^{∘∘}2 (concentration=18 μM) showed a stabilizing effect on miR-21 under these conditions resulting in residual RNA levels at 20% FBS to be comparable to those at 1% FBS in the absence of peptide (FIG. 14c, left). In comparison, the stabilizing effect of wt33 was considerably smaller while monomeric peptide 3 did not have an effect under these conditions (FIGS. 14a and 14b). Both is in line with their reduced affinity for miR-21 (FIGS. 12a and 12b). To investigate the time-dependence of RNA stability, a prolonged incubation of RNA in serum (27.5% FBS) was performed, and the remaining RNA visualized by native PAGE (FIG. 14d). Remarkably, even after 6 hours, miR-21 was still detectable in the presence of peptide 2^{∘∘}2, while in the absence of the peptide, RNA was fully degraded within 15 minutes in line with afore made observations. These findings highlight the potential of peptide 2^{∘∘}2 to strongly enhance the stability of dsRNA in serum.

In summary, applicants have shown that the compounds according to the present invention enhance the delivery of duplex RNA into cells. Applicants thus generated peptides whose dsRNA-binding affinity was tuneable through all-hydrocarbon stapling and covalent dimerization via N-terminal disulfide bridges. Notably, dimerization enhanced the stability of peptide/dsRNA complexes but also promoted their cellular uptake. Illustrating the stimuli-responsive nature of applicants' design, complexes formed with either peptide dimer 1^{∘∘}1 or 2^{∘∘}2 were susceptible to disassembly once treated with excess reducing agents. This observation was also extended to cellular permeability, where treatment with reducing agent resulted in reduced cellular uptake of siRNA, thereby providing a platform technology for peptide-based siRNA carriers for RNA-specific peptide ligands for RNA delivery.

### Materials and Methods: Oligonucleotides

The sequences and names of all oligonucleotides are presented in Table 3. High-performance liquid chromatography (HPLC)-purified oligonucleotides were used. For quantification, the ultraviolet (UV) absorbance of the oligonucleotides was measured in the buffer of the corresponding experiment using a Nanodrop One UV/Vis spectrophotometer (Thermofisher). Respective concentrations were calculated with a molar extinction coefficient at λ=260 nm, determined according to the nearest neighbour model using published parameters for oligonucleotides. RNA duplexes were heated to 95° C. for 10 min and slowly cooled to room temperature (RT) for 1 h prior to experiments. For hairpins, RNA was snap cooled on ice instead.

### Solid-Phase Peptide Synthesis

Wt33 was synthesized according to previously reported procedures. All other peptides were synthesized according to the following protocols on H-Rink amide ChemMatrix® resin (Sigma Aldrich, loading 0.4 mmol/g) using an Fmoc-based solid-phase peptide synthesis strategy.

### Automated Peptide Synthesis

Automated peptide synthesis was performed using a Syro I (MultiSynTech). Synthesis followed a deprotect, couple, cap workflow. Fmoc-protected amino acids were prepared as 0.33 M solutions dissolved in 0.33 M Oxyma (DMF) and coupling reagents were dissolved in DMF (c=0.33 M). DIPEA was dissolved in NMP (c=1.33 M). Dry resin was typically swollen in DMF for at least 30 minutes before automated synthesis. Between each reaction step, resins were washed with 6 syringe volumes of DMF. Fmoc deprotection was carried out in Piperidine/DMF (1/5, v/v), 2×5 min. Coupling of amino acids was performed as double couplings, Fmoc-aa-OH (4 eq.), PyBOP/HATU (3.9 eq.) and DIPEA (c=1.33 M) for 30 minutes each. After each double coupling cycle, resins were treated with Ac2O/NMP (1/10, v/v), 2×5 min.

### Manual Peptide Synthesis

All reaction steps were performed at room temperature in syringe reactors. Resins were suspended by shaking syringe reactors on an orbital shaker. Synthesis followed a deprotect, couple, cap workflow. Dry resin was typically swollen in DMF for 30 minutes before an initial reaction. In between reaction steps, resins were washed with DMF (3×, 1 mL per 50 mg of resin), DCM (3×, 1 mL per 50 mg of resin) and DMF (3×, 1 mL per 50 mg of resin).

### Fmoc Deprotection

Resins were treated with a solution of Piperidine/DMF (1/5, v/v, 1 mL per 50 mg of resin) for 2×10 min.

### Manual Amino Acid Coupling Procedure

Fmoc-aa-OH (4 eq.) was prepared with Oxyma (4 eq.) and COMU (4 eq.) in DMF (c=0.25 M) and activated with DIPEA (8 eq.). The coupling solution was added to the resin and shaken at RT for 30 minutes. The solution was subsequently discarded, the resin washed and then treated with a second coupling solution composed of Fmoc-aa-OH (4 eq.), Oxyma (4 eq.) and PyBOP (4 eq.) in DMF (c=0.25 M) which was activated with DIPEA (8 eq.). Resins were shaken for 30 minutes at before the coupling solution was discarded.

### N-Acetylation (Capping)

Free amino groups were acetylated by treating resins with a solution of Ac20/DIPEA/DMF (1/1/8, v/v/v, 1 mL per 50 mg of resin) for 2×5 min.

### Ring-Closing Metathesis (RCM)

After synthesis of the core peptide sequence, resins were first swollen in dry DCE for 30 minutes before performing ring-closing olefin metathesis (RCM). To begin the reaction, a solution of Grubbs 1^{st} generation catalyst in dry DCE of (4 mg/mL, 1 mL per 50 mg resin) was added to the resin and a continuous stream of nitrogen was bubbled through the reaction mixture. After 1 hour, the reaction solution was discarded and the resin was washed with dry DCE (3×, 1 mL per 50 mg of resin). This procedure was repeated an additional three times, before the resin was washed with a DCM/DMSO-solution (1/1, v/v) for 10 min and subsequently with DCM (3×).

### Cleavage, Purification, and Characterization

Before final cleavage, the resin was dried under vacuum. A solution of TFA/thioanisole/H₂O/EDT (87.5/5/5/2.5, v/v/v/v, 2 mL/20 μmol resin) was added to the resin for 4×1 h. The cleavage solution was then partially evaporated followed by the addition of cold diethyl ether to precipitate the crude peptide. After centrifugation (4° C., 4000 rpm, 15 min), the supernatant was discarded, the crude product was dissolved in H₂O/ACN (5/1, v/v) and lyophilized. Crude lyophilised peptides were re-dissolved in H₂O/ACN (19/1, v/v) and purified by reversed-phase HPLC (Column: Macherey-Nagel Nucleodur C18, 10×125 mm, 110 Å, 5 μm. Solvent A: H2O+0.1% TFA Solvent B: ACN+0.1% TFA. Flow Rate: 6 mL/min). An isocratic gradient from 5-30% Solvent B over 40 minutes was typically used for peptide purification. Pure fractions were subsequently pooled and lyophilized followed by characterization and quantification. Peptides were characterized using an analytical reversed-phase HPLC (1260 Infinity, Agilent Technology. Column: Agilent Eclipse XDB-C18, 4.6×150 mm, 5 μm. Solvent A: H₂O+0.1% TFA, Solvent B: ACN+0.1% TFA. Flow Rate: 1 mL/min, 5-65% gradient over 30 minutes) coupled to an ESI-MS (6120 Quadrupole LC/MS, Agilent Technology).

Analytical HPLC chromatograms at 210 nm and MS spectra (masses and m/z ratios in Table 4) are provided in FIGS. 5 and 6. To prepare stocks for quantification and follow-up experiments, lyophilized peptides were re-dissolved in nuclease free water. Peptides were quantified by HPLC-based comparison (λ=210 nm) with reference to a gravimetrically-quantified peptide standard.

### Peptide Dimerization

Dimerization was carried out by diluting thiolated peptide stocks (c=1 mg/mL) in 0.1 M ammonium bicarbonate buffer (pH=8.0) and allowing to stir for 20 hours. The reaction was monitored by analytical reversed-phase HPLC (1260 Infinity, Agilent Technology. Column: Agilent Eclipse XDB-C18, 4.6×150 mm, 5 μm. Solvent A: H₂O+0.1% TFA, Solvent B: ACN+0.1% TFA. Flow Rate: 1 mL/min, 10-30% gradient over 10 minutes) coupled to an ESI-MS (6120 Quadrupole LC/MS, Agilent Technology). Upon completion, the reaction solution was lyophilized and then re-dissolved in H₂O/CAN (19/1, v/v) before being purified using the same reversed-phase HPLC procedure described in the previous section.

### Electrophoretic Mobility Shift Assay (EMSA)

Electrophoretic mobility shift assays (EMSA's) were performed using a Bio-Rad MiniProtean gel system paired with a direct current (DC) power source (PowerPac™ HC, BioRad). Typically, 6 μL solutions containing RNA (concentration=3 μM) and peptide (concentration=6 μM) were incubated for 1 h at RT in a binding buffer (1×TAE and 10% glycerol). For gels monitoring disulfide reduction, peptide/RNA solutions were incubated in increasing concentrations (concentration=6, 60 and 600 μM) of TCEP. After incubation, bound nucleic acid complexes were resolved using 15% and 10% non-denaturing polyacrylamide gels (acrylamide:bis-acrylamide (19:1) in 1×TAE) at 150 V or 120 V in running buffer (1×TAE) at 4° C. for 1.5 hours. For nucleic acid visualization, gels were stained using 2 μL of SYBR™ gold nucleic acid gel dye (Invitrogen) in 20 mL of 1×TAE buffer for 15 minutes at RT before being imaged using a Bio-Rad ChemiDoc.

### Circular Dichroism (CD) Spectroscopy & Tₘ Determination

Circular dichroism (CD) spectra were recorded with a Jasco J-1500 spectropolarimeter (Jasco) equipped with a programmable Peltier thermostat in a stoppered quartz cuvette (10 mm; Hellma). Samples were prepared in a buffer of 10 mM sodium phosphate and 100 mM NaCl (pH=7.4). For measurements of individual species, 2 μM solutions were prepared. For measurements of co-incubated peptide/oligonucleotide species, equimolar solutions (concentration=2 μM) were prepared. Samples co-incubated with TCEP were prepared in a buffer of 10 mM sodium phosphate, 100 mM NaCl and 1 mM TCEP (pH=7.4) and allowed to incubate for 1 h at room temperature before measurement. For each sample, 10 CD spectra were measured between 200 nm and 350 nm with continuous scan mode (1 mdeg sensitivity, 1.0 nm resolution, 1.0 nm bandwidth, 2 s integration time, 100 nm/min scan rate). Obtained spectra were averaged and then subtracted from a reference buffer spectrum. CD data were normalized to oligonucleotide strand concentration using Formula 1:

$$ε l - ε r = Δ ε = \frac{θ}{32980 \cdot c \cdot l} \qquad ( 1 )$$

where θ=observed ellipticity/mdeg, c=DNA or RNA strand concentration/mol/L and l=path length/cm.
Melting temperature (Tₘ) determination was conducted with the same instrumentation and sample preparation, where ellipticity (θ=267 nm) was measured by ramping the temperature from 15-90° C. (4° C./minute ramp, ±0.05° C. equilibration tolerance, 6 seconds delay after equilibration). Points were taken every 0.5° C. Raw data were normalized as described above, and Tm-values were determined using the CDpal program before being plotted in Prism 5.0 (GraphPad).

### Confocal Microscopy of HEK Cells

HEK cells were seeded at a density of 40000 cells per well on an 8 well micro-slide (Ibidi), one day before the experiment. They were cultured in DMEM (Thermofisher Scientific), supplemented with 10% Fetal Calf Serum (FCS, PAN Biotech) and glutamax (Life technologies). Before starting the experiment, the peptides and Cy5-labelled siRNA were incubated at a peptide/siRNA ratio of 2:1 (6 μM-3 μM) for 1 h. In case for dithiothreitol (DTT) supplemented experiments, DTT was added to a concentration of 20 mM. The samples were diluted to a final siRNA concentration of 1 μM in serum free DMEM. Cells were washed 1× to remove FCS, and incubated in the siRNA:peptide solution for 1 hour at 37° C. After incubation, cells were washed with the phenol-red free DMEM with HEPES, and imaged. Cy5-labelled siRNA uptake was imaged with an SP5 Laser Scanning Confocal Microscope (Leica) on a temperature-controlled stage at 37° C. An HCX PL APO 63×1.2 with water immersion lens was used. A HeNe laser line at 633 nm was used for excitation, the detection window of the PMT was set between 680 nm and 700 nm. Images were visualized and processed in Fiji.

### Isothermal Titration Calorimetry (ITC)

Isothermal titration calorimetry (ITC) was conducted using a VP-ITC (MicroCal). Before measurement, annealed miR-21 and peptide samples were dissolved in 1×PBS and degassed using a ThermoVac and heated to 30° C. miR-21 (c=6-15 μM) was then transferred into the sample cell (1.8 mL) and peptides (c=86-305 μM) were transferred into the syringe (300 μL). 37 Injections per measurement were performed at 30° C. (8 μl injection volume, 3.2 s injection time, 180 s spacing, high feedback mode) with an initial delay of 60 s and a stirring speed of 310 rpm. Data were analyzed using the MicroCal LLC ITC software (Origin; OriginLab Corporation), the heat associated with each injection was calculated by integrating the area under the curve for each injection and then normalized to concentration. A ‘single set of identical binding sites’ model was used to fit the binding curves from which thermodynamic binding parameters (ΔG, ΔH, ΔS, N and Kd) were calculated.

### Disulfide Cleavage Assay

The reduction properties of the disulfide bond were determined by incubating peptide 2^{∘∘}2 with a physiological relevant concentration of glutathione (GSH) with and without miR-21. First, 200 μL of a 125 μM peptide 2^{∘∘}2 solution and, if necessary, 62.5 μM miR-21 were prepared in water. For t=0, 36 μL (4.5 nmol 2^{∘∘}2) of the assay solutions were picked and topped up with ddH2O to 90 μL followed by addition of 10 μL TFA. The remaining assay solutions were diluted with 141 μL ddH2O, 81.3 μL phosphate buffer (0.5 M, pH=8.0) and 20.4 μL of an aqueous glutathione solution (100 mM, pH=8.0). The addition of glutathione started the assay and probes were taken at 5, 10, 15 and 30 min by picking 90 μL (4.5 nmol 2^{∘∘}2) of the assay solution followed by adding 10 μL of TFA to stop the reaction. The reduction was monitored by injecting 95 μL of the samples to a LC-MS system (the above mentioned analytical reversed-phase HPLC coupled to an ESI-MS with a gradient of Solvent A: H₂O+0.1 formic acid and 0.01% TFA, Solvent B: ACN+0.1 formic acid and 0.01% TFA, Flow Rate: 1 mL min-1, from 0-18.6% B in 30 min). The measured spectra were evaluated with MestreNova 11.0.

### Stabilization Assay

RNA stabilization was investigated by incubation of peptide/RNA complexes with fetal bovine serum (FBS) (Thermo Fischer Scientific). Peptide/RNA complexes were formed as described above after which defined amounts of FBS were added (concentration(RNA)=9 μM, concentration(peptide)=18 μM, total volume 10 μL). For gradient stability assay FBS containing RNA solutions were incubated at 37° C. for 10 min and for time course analysis at room temperature with 27.5% FBS. After incubation, 10 μL of NaOAc (3 M), 1 μL Glycogen (Thermo Fischer Scientific), 1 μL Ribolock (Thermo Fischer Scientific) and ddH2O was added on ice to a total volume of 200 μL.

RNA was extracted twice with freshly prepared solution of phenol/chloroform (5:1, 2 mL). After ethanol precipitation (1 mL) of the aqueous phase RNA pallets were redissolved in 8 μL ddH2O and added 2 μL 6×BB and then loaded into a native PAGE gel and run with previously described conditions.

Table 3 shows an overview of oligonucleotides tested with corresponding 5′ modifications, sequence (from 5′-end to 3′-end, left to right, 1-letter code), length and molecular weight (MW in g/mol). All oligonucleotides were synthesized with 3′ hydroxyl groups. Modifications: P=5′-terminal phosphate, Cy5=Cyanine 5: 2-((1E,3E)-5-((E)-1-(3-(λ-oxidaneyl)propyl)-3,3-dimethylindolin-2-ylidene)penta-1,3-dien-1-yl)-1-(3hydroxypropyl)-3,3-dimethyl-3H-indol-1-ium.

TABLE 3

| | 5′ | | | MW | SEQ ID |
|---|---|---|---|---|---|
| Oligonucleotide | Mod. | Sequence (5′ - 3′) | Length | (g/mol) | NO |
| miR-21 5′ | P | UAG CUU AUC AGA CUG | 22 | 7084.2 | 15 |
| | | AUG UUG A | | | |
| miR-21 3′ | P | AAC ACC AGU CGA UGG | 20 | 6791.1 | 16 |
| | | GCU GU | | | |
| Cy5 siRNA 5′ | Cy5 | CUU ACG CUG AGU ACU | 21 | 7249.4 | 17 |
| | | UCG ATT | | | |
| Cy5 siRNA 3′ | P | UCG AAG UAC UCA GCG | 21 | 6692.9 | 18 |
| | | UAA GTT | | | |

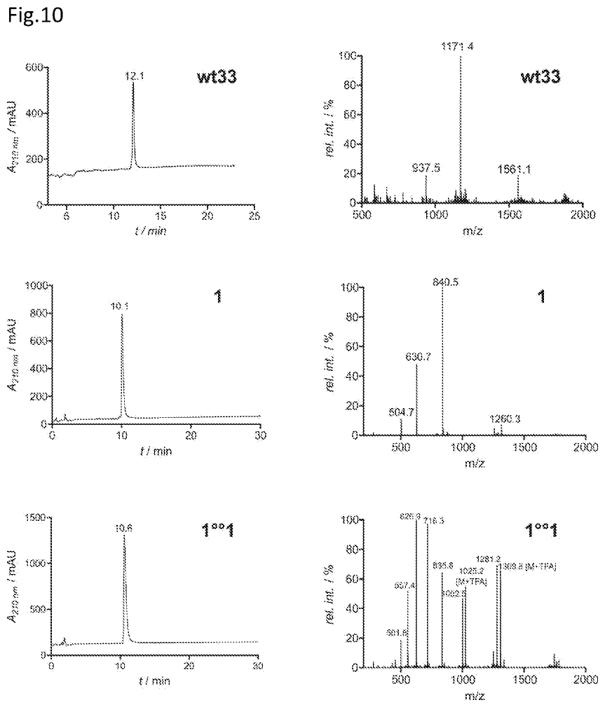

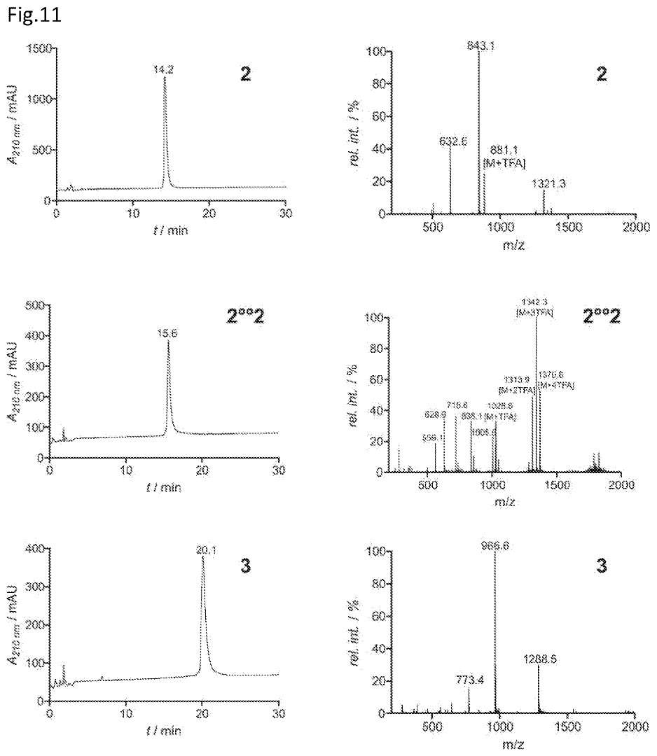

Table 4 shows an overview of all synthesized peptides with corresponding N-terminal modification, sequence (from N- to C-terminus, left to right, 1-letter code), calculated mass-to-charge ratios (m/z calc.) and found masses (m/z found) for charged ions ([M+nH] n+). Ac=acetyl, S—S=N-terminal disulfide, β=beta-alanine, X=3-mercaptopropionic acid, S5=(S)-2-(4-pentenyl) alanine, for compound 1, Dimer 1^{∘∘}1; compound 2, Dimer 2^{∘∘}2; and comparative compounds not having a dimerization motif wt33 and 3. HPLC/MS analysis of these compounds can be found in FIG. 10 and FIG. 11.

TABLE 4

| | N- | | | | SEQ |
|---|---|---|---|---|---|
| | term. | | m/z | m/z | ID |
| Peptide | mod. | Sequence (N -C) | calc. | found | NO |
| wt33 | Ac | KKQAQRKRHKLNRKERGHKSPSEQR | 1048.2 | 1048.1 | 19 |
| | | RSELWHAR | | [M + 4H]⁴⁺ | |
| 1 | Ac | MNQKKQAQRKRHKLNRKER | 840.4 | 840.5 | 20 |
| | | | | [M + 3H)³⁺ | |
| 1ºº1 | S-S | XβNQKKQAQRKRHKLNRKER | 626.8 | 626.9 | 21 |
| | | \| | | [M + 8H]⁸⁺ | |
| | | XβNQKKQAQRKRHKLNRKER | | | |
| 2 | Ac | MNQKKQAQRKRHKS₅NRKS₅R | 843.2 | 843.1 | 22 |
| | | | | [M + 3H]³⁺ | |
| 2ºº2 | S-S | XβNQKKQAQRKRHKS₅NRKS₅R | 718.5 | 718.6 | 23 |
| | | \| | | [M + 7H]⁷⁺ | |
| | | XβNQKKQAQRKRHKS₅NRKS₅R | | | |
| 3 | Ac | PLHEIIRKLERMNQKKQAQRKRHKLN | 966.5 | 966.6 | 24 |
| | | RKER | | [M + 4H]⁴⁺ | |

1-36. (canceled)

37. A peptide-based compound comprising a structure p-x-b-x′-p′, wherein
i each of p and p′ is independently an oligonucleotide-binding motif;
ii each of x and x′ is independently an optional linker motif, and
iii b is a linking motif coupling the oligonucleotide-binding motifs to form a dimerized form,
wherein each of motif p and p′ is independently a peptide chain having the following fragment comprising a contiguous sequence of at least 14 amino acid residues and having the following general sequence (I),
wherein the N-terminal position 1 is located on the left side: 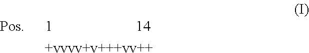
wherein “v” is a variable amino acid residue position,
wherein “+” is a position with a positively charged amino acid residue,
wherein the contiguous sequences of p and p′ are selected from a sequence comprising at least the first 14 amino acids counted from the N-terminus of general Seq. No 1a to general Seq. No 1 m, which may be varied at positions by additional side chain-to-side chain crosslinking amino acids “#”, and possible combinations thereof, or a functional variant thereof having at least 90% identity thereof,
wherein the N-terminal position 1 is located on the left side:

| Pos. | 1 32 | | |
|---|---|---|---|
| | KKQAQRKRHKLNRKERGHKSPSEQRRSELWHA | (Seq. No 1a); | (SEQ ID NO 1) |
| | KKQ#QRKRHK#NRKERGHKSPSEQRRSELWHA | (Seq. No 1b); | (SEQ ID NO 2) |
| | KKQAQR#RHK#NRKERGHKSPSEQRRSELWHA | (Seq. No 1c); | (SEQ ID NO 3) |
| | KKQAQRKRHK#NRK#RGHKSPSEQRRSELWHA | (Seq. No 1d); | (SEQ ID NO 4) |
| | KKQAQRKRHKLNRKERGHKSPSE#RRS#LWHA | (Seq. No 1e); | (SEQ ID NO 5) |
| | KKQAQRKRHKLNRKERGHKSPSEQRRS#LWH# | (Seq. No 1f); | (SEQ ID NO 6) |
| | KKQ#QRKRHK#NRKERGHKSPSE#RRS#LWHA | (Seq. No 1g); | (SEQ ID NO 7) |
| | KKQ#QRKRHK#NRKERGHKSPSEQRRS#LWH# | (Seq. No 1h); | (SEQ ID NO 8) |
| | KKQAQR#RHK#NRKERGHKSPSE#RRS#LWHA | (Seq. No 1i); | (SEQ ID NO 9) |
| | KKQAQR#RHK#NRKERGHKSPSEQRRS#LWH# | (Seq. No 1k); | (SEQ ID NO 10) |
| | KKQAQRKRHK#NRK#RGHKSPSE#RRS#LWHA | (Seq. No 1l); | (SEQ ID NO 11) |
| and | | | |
| | KKQAQRKRHK#NRK#RGHKSPSEQRRS#LWH# | (Seq. No 1m), | (SEQ ID NO 12) |

and
wherein the dimerization motif b comprises a cleavable link and/or wherein the motif b covalently connects the oligonucleotide-binding motifs.

38. The compound according to claim 37, wherein each of motif p and p′ is independently a peptide chain having a fragment comprising a contiguous sequence of at most 32 amino acid residues, and having the following general sequence (II), and
wherein the N-terminal position 1 is located on the left side:

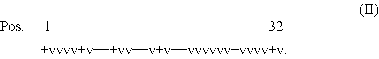

39. The compound according to claim 37, wherein each of motif p and p′ is independently a peptide chain having a fragment comprising at most a contiguous sequence of 32 amino acid residues, and having the following general sequence (III), wherein the N-terminal position 1 is located on the left side: 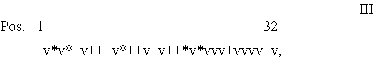
wherein “*” comprises a natural and non-natural polar amino acid residue selected from Glu, Asn, and Ser.

40. The compound according to claim 37, wherein the contiguous sequences of p and p′ are selected from general Seq. No 1a to general Seq. No 1m:

| | | | |
|---|---|---|---|
| Pos. | 1 32 | | |
| | KKQAQRKRHKLNRKERGHKSPSEQRRSELWHA | (Seq. No 1a); | (SEQ ID NO 1) |
| | KKQ#QRKRHK#NRKERGHKSPSEQRRSELWHA | (Seq. No 1b); | (SEQ ID NO 2) |
| | KKQAQR#RHK#NRKERGHKSPSEQRRSELWHA | (Seq. No 1c); | (SEQ ID NO 3) |
| | KKQAQRKRHK#NRK#RGHKSPSEQRRSELWHA | (Seq. No 1d); | (SEQ ID NO 4) |
| | KKQAQRKRHKLNRKERGHKSPSE#RRS#LWHA | (Seq. No 1e); | (SEQ ID NO 5) |
| | KKQAQRKRHKLNRKERGHKSPSEQRRS#LWH# | (Seq. No 1f); | (SEQ ID NO 6) |
| | KKQ#QRKRHK#NRKERGHKSPSE#RRS#LWHA | (Seq. No 1g); | (SEQ ID NO 7) |
| | KKQ#QRKRHK#NRKERGHKSPSEQRRS#LWH# | (Seq. No 1h); | (SEQ ID NO 8) |
| | KKQAQR#RHK#NRKERGHKSPSE#RRS#LWHA | (Seq. No 1i); | (SEQ ID NO 9) |
| | KKQAQR#RHK#NRKERGHKSPSEQRRS#LWH# | (Seq. No 1k); | (SEQ ID NO 10) |
| | KKQAQRKRHK#NRK#RGHKSPSE#RRS#LWHA | (Seq. No 1l); | (SEQ ID NO 11) |
| | KKQAQRKRHK#NRK#RGHKSPSEQRRS#LWH# | (Seq. No 1m). | (SEQ ID NO 12) |

41. The compound according to claim 40, wherein a fragment p or p′ comprising two motifs “#”-“#” comprises one or more of the complementary substituents a) to g) prior to cyclisation: 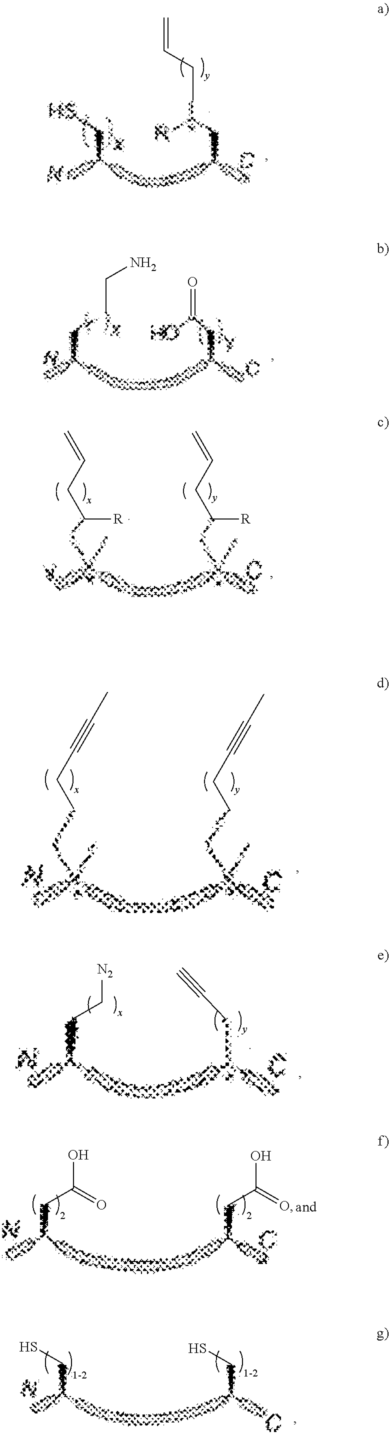
wherein x and y are integers in the range of from 1 to 5,
wherein R is hydrogen, an optionally substituted C₁-C₆-alkyl, an optionally substituted C₁-C₆-alkenyl, or an optionally substituted C₁-C₆ alkynyl; and
wherein a fragment p or p′ comprises two linked motifs “#”-“#” i to ix, linked either by the cyclisation of a) to e), or from the insertion reaction of f) with vi to form vii, or from the insertion reaction of g) with viii to form ix, respectively:

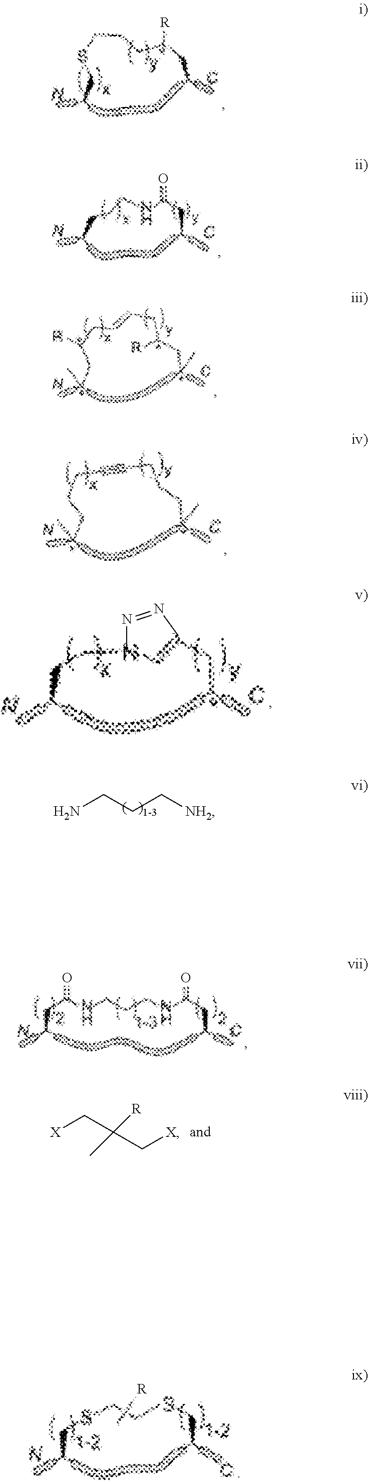

42. The compound according to claim 41, wherein the contiguous sequences of p and p′ are selected from general Seq. No 1n to general Seq. No 1y:

| | | | |
|---|---|---|---|
| Pos. | 1 14 26 | | |
| | KKQAQRKRHKLNRKER | Seq. No 1n | SEQ ID NO 13 |
| | KKQäQRKRHKöNRKER | Seq. No 1o | SEQ ID NO 25 |
| | KKQAQRöRHKöNRKER | Seq. No 1p | SEQ ID NO 26 |
| | KKQAQRKRHKöNRKöR | Seq. No 1q | SEQ ID NO 27 |
| | KKQAQRKRHKLNRKERGHKSPSEöRRSö | Seq. No 1r | SEQ ID NO 28 |
| | KKQAQRKRHKLNRKERGHKSPSEQRRS | Seq. No 1s | SEQ ID NO 29 |
| | KKQäQRKRHKöNRKERGHKSPSEöRRSö | Seq. No 1t | SEQ ID NO 30 |
| | KKQäQRKRHKöNRKERGHKSPSEQRRS | Seq. No 1u | SEQ ID NO 31 |
| | KKQAQRöRHKöNRKERGHKSPSEöRRSö | Seq. No 1v | SEQ ID NO 32 |
| | KKQAQRöRHKöNRKERGHKSPSEQRRS | Seq. No 1w | SEQ ID NO 33 |
| | KKQAQRKRHKöNRKöRGHKSPSEöRRSö | Seq. No 1x | SEQ ID NO 34 |
| | KKQAQRKRHKöNRKöRGHKSPSEQRRS | Seq. No 1y | SEQ ID NO 35 |

wherein, ä is 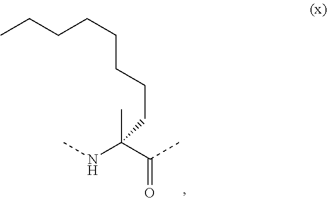
and ö is 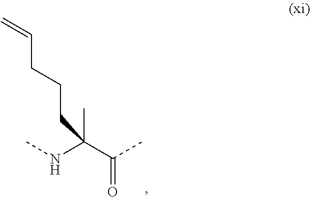
forming the bracket 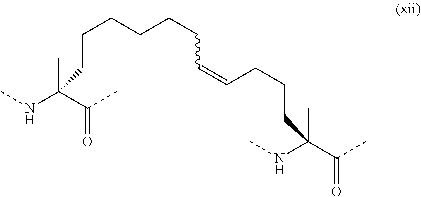
after crosslinking;
or each of ö and ö is 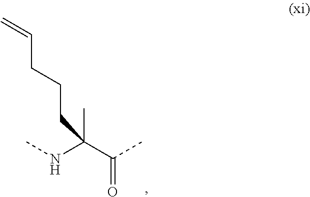
forming the bracket 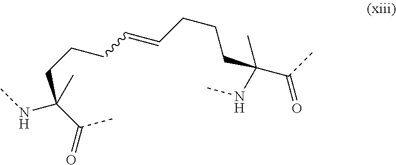
after crosslinking.

43. The compound according to claim 37, wherein the motif b comprises a covalent bond sensitive to reduction, radiation and/or enzymatic digestion.

44. The compound according to claim 43, wherein the motif b comprises a disulfide bridge connecting the N-terminal amino acid residues of p and p′, or of x and x′; or
wherein the motif b comprises a linker composed of thiol-substituted amino acids covalently bonded through a disulfide bride, according to the general structure (xiv) 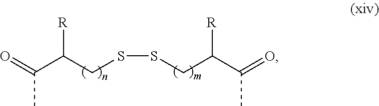
wherein:
n and m each independently is an integer from 1 to 4; and
R is hydrogen, a substituted or unsubstituted alkyl, substituted or unsubstituted alkyl, substituted or unsubstituted heteroalkyl, a substituted or unsubstituted aryl, —NH₂, —N(H)CH₃COOH, or an amide selected from C₂ to C₁₂ aliphatic, optionally alkylated, amidated, or acylated carboxylic acids.

45. The compound according to claim 37, wherein the optional linkers x and x′ each independently are selected from polar amino acid residues, peptides, or —(OCH₂CH₂)_{z}— polyethylene glycol-based linkers, wherein z is an integer from 1 to 50.

46. The compound according to claim 37, wherein the entire compound is a homodimer, or a heterodimer.

47. The compound according to claim 37, wherein the motif of p and p′ comprises the amino acid sequence of SEQ ID NO 13; or wherein the motif p or p′ each consists of the amino acid sequence of SEQ ID NO 13, or a functional variant thereof having at least 85%, 90%, or 95% identity to any one of SEQ ID NO 13.

48. The compound according to claim 37, wherein each peptide chain p-comprises a minimum length of at least 14, 15, 16, 17, 18, 19, 20, 21, 22, 23, 24, 25, 26, 27, 28, 29, 30, 31 and up to and including 32 amino acid residues selected from natural or non-natural amino acid residues.

49. The compound according to claim 37, wherein motif p an p′ each comprises a helix-forming peptide sequence, wherein the helix-forming peptide sequence comprises at least 50% of positively charged amino acids,
wherein the compound is convertible into two non-bonded oligonucleotide-binding motifs p and p′, and wherein the conversion results in reduced affinity of the complex for the double-stranded oligonucleotide; or
wherein the oligonucleotide-binding motif comprises a cyclic bracket to enhance its binding conformation.

50. The compound according to claim 37, wherein the compound comprises or consists of the amino acid sequence of SEQ ID NO 14, or a functional variant thereof having at least 85%, 90%, or 95% identity to any one of SEQ ID NO 14.

51. The compound according to claim 37, having the general structure xvii: 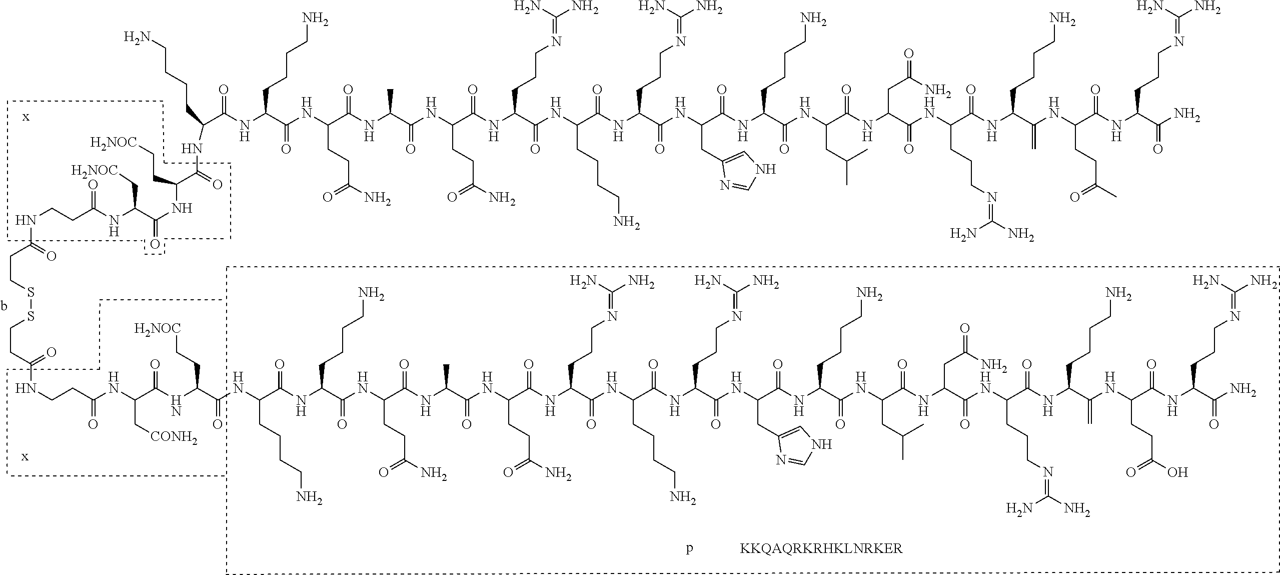
(xvii); or having the general structure xviii: 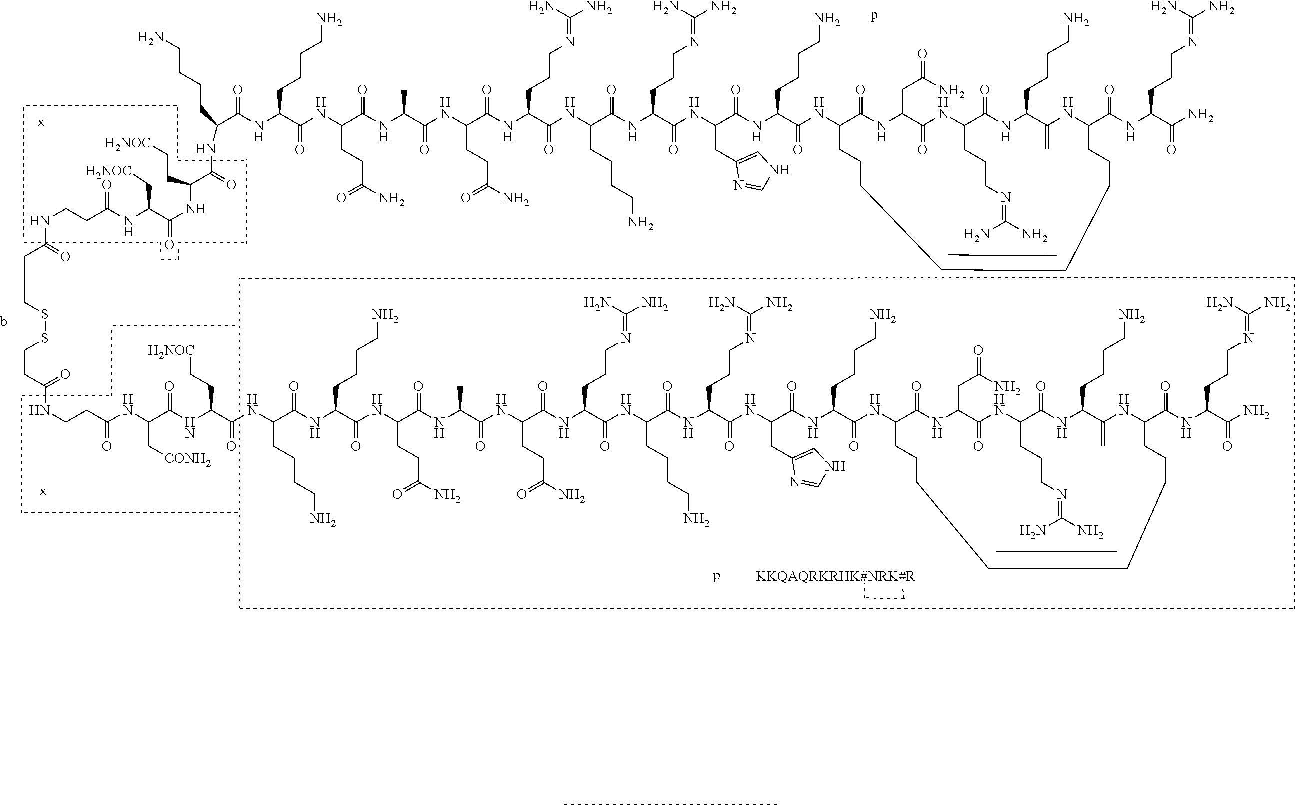
(xviii).

52. A monomeric compound for forming a dimeric compound according to claim 37, comprising a structure p-x-a, wherein:
i p is an oligonucleotide-binding motif;
ii x is an optional linker motif, and
iii a is a linkable motif capable of coupling the compound to an identical or different compound to, form a homo- or heterodimer;
wherein motif p is a peptide chain having the following fragment comprising a contiguous sequence of at least 14 amino acids, and having the following general sequence (I), wherein the N-terminal position 1 is located on the left side: 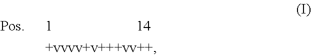
wherein the contiguous sequence of p is selected from a sequence comprising at least the first 14 amino acids counted from the N-terminus of general Seq. No 1a to general Seq. No 1m, which may be varied at positions by additional side chain-to-side chain crosslinking amino acids “#”, and possible combinations thereof, or a functional variant thereof having at least 90% identity thereof,
wherein the N-terminal position 1 is located on the left side:

| | | | |
|---|---|---|---|
| Pos. | 1 32 | | |
| | KKQAQRKRHKLNRKERGHKSPSEQRRSELWHA | (Seq. No 1a); | (SEQ ID NO 1) |
| | KKQ#QRKRHK#NRKERGHKSPSEQRRSELWHA | (Seq. No 1b); | (SEQ ID NO 2) |
| | KKQAQR#RHK#NRKERGHKSPSEQRRSELWHA | (Seq. No 1c); | (SEQ ID NO 3) |
| | KKQAQRKRHK#NRK#RGHKSPSEQRRSELWHA | (Seq. No 1d); | (SEQ ID NO 4) |
| | KKQAQRKRHKLNRKERGHKSPSE#RRS#LWHA | (Seq. No 1e); | (SEQ ID NO 5) |
| | KKQAQRKRHKLNRKERGHKSPSEQRRS#LWH# | (Seq. No 1f); | (SEQ ID NO 6) |
| | KKQ#QRKRHK#NRKERGHKSPSE#RRS#LWHA | (Seq. No 1g); | (SEQ ID NO 7) |
| | KKQ#QRKRHK#NRKERGHKSPSEQRRS#LWH# | (Seq. No 1h); | (SEQ ID NO 8) |
| | KKQAQR#RHK#NRKERGHKSPSE#RRS#LWHA | (Seq. No 1i); | (SEQ ID NO 9) |
| | KKQAQR#RHK#NRKERGHKSPSEQRRS#LWH# | (Seq. No 1k); | (SEQ ID NO 10) |
| | KKQAQRKRHK#NRK#RGHKSPSE#RRS#LWHA | (Seq. No 1l); | (SEQ ID NO 11) |
| | and | | |
| | KKQAQRKRHK#NRK#RGHKSPSEQRRS#LWH# | (Seq. No 1m), | (SEQ ID NO 12) |

and
wherein “v” represents a variable amino acid residue position, and wherein “+” represents a position with a positively charged amino acid.

53. The compound according to claim 52, comprising a contiguous sequence of at most 32 amino acid residues, and having the following general sequence (II):
Pos. 1 32
